(19) United States
(12) Patent Application Publication
(10) Pub. No.: US 2026/0021092 A1
(43) Pub. Date: Jan. 22, 2026

(54) STRUCTURE-BASED DESIGN OF A NOVEL CLASS OF DRUGS FOR NEURODEGENERATIVE DISEASE

(71) Applicant: The Regents of the University of California, Oakland, CA (US)

(72) Inventors: David S. Eisenberg, Los Angeles, CA (US); Paul M. Seidler, Los Angeles, CA (US); Kevin A. Murray, Los Angeles, CA (US); David R. Boyer, Santa Monica, CA (US); Jiahui Lu, Los Angeles, CA (US)

(73) Assignee: The Regents of the University of California, Oakland, CA (US)

(21) Appl. No.: 18/998,707

(22) PCT Filed: Aug. 16, 2023
(86) PCT No.: PCT/US23/72275

§ 371 Date: Jan. 27, 2025

Related U.S. Application Data
(60) Provisional application No. 63/398,367, filed on Aug. 16, 2022.

(51) Int. Cl. A61K 31/502; A61K 31/167; A61K 31/437; A61P 25/28; G01N 33/50; G16B 15/30; G16B 35/00

(52) U.S. Cl. CPC A61K 31/502; A61K 31/167; A61K 31/437; A61P 25/28; G01N 33/5038; G16B 15/30; G16B 35/00

(57) ABSTRACT

The invention provides pharmaceutical compositions comprising molecules capable of disassembling tau amyloid fibrils or alpha-synuclein amyloid fibrils, as well as methods for making compositions comprising these molecules. Embodiments of the invention also include methods for using these molecules to facilitate the disassembling of tau amyloid fibrils or alpha-synuclein amyloid fibrils.

### CROSS-REFERENCE TO RELATED APPLICATIONS

This application claims priority under Section 120 from U.S. Patent Application Ser. No. 63/398,367, filed Aug. 16, 2022, the contents of which are incorporated herein by reference.

### STATEMENT REGARDING FEDERALLY SPONSORED RESEARCH AND DEVELOPMENT

This invention was made with government support under Grant Numbers NS095661, AG054022 and AG029430, awarded by the National Institutes of Health. The government has certain rights in the invention.

### TECHNICAL FIELD

The present invention relates to compounds that bind amyloid fibrils and methods for making and using them.

### BACKGROUND OF THE INVENTION

Recognizing that Alzheimer's disease (AD) is the most common neurodegenerative disease worldwide, increasingly prevalent in our aging population, scientists have sought therapeutic interventions capable of slowing disease progression. Although cognitive decline and brain atrophy correlate with tau fibril formation in AD¹⁻³, finding effective therapeutics that enter the brain, enter neurons that house tau fibrils and break down the fibrils remains a challenge. Considerable efforts have been made to diminish tau aggregation in AD, including stabilizing microtubules, inhibiting tau phosphorylation or acetylation, reducing tau expression, or inhibiting fibrilization⁴. Tau aggregation inhibitors including curcumin and methylene blue/LMTX have progressed to Phase II and Phase III clinical trials, respectively, but have yet to show efficacy at treating disease⁵. Here we pursue a new approach. We introduce structure-based screening to identify small molecules capable of disaggregating tau amyloid fibrils, as a first step on a new therapeutic route for Alzheimer's disease.

Despite the unusual stability of pathogenic amyloid fibrils, most robustly stable in SDS, urea, guanidinium, and at elevated temperatures, a few small molecules are known to disaggregate fibrils in aqueous conditions into smaller non-toxic species. Prominent among these is epigallocatechin gallate (EGCG) a polyphenolic compound found in green tea and extensively investigated with similar compounds in some 4000 research papers⁶⁻¹⁴. However, EGCG has not proven an effective drug, perhaps because of limited bioavailability (particularly in brain), promiscuous protein binding, and ready modification in bodily fluids¹⁵. Unlike other small molecules that inhibit tau fibril formation, EGCG disaggregates previously formed fibrils, indicating that visualization of EGCG bound to the fibrils may be possible before disaggregation.

In addition, Parkinson's disease (PD), multiple system atrophy (MSA), and dementia with Lewy bodies (DLB) are neurodegenerative disorders characterized by abnormal accumulation of the protein alpha-synuclein (uSyn). Known as synucleinopathies, these diseases are hallmarked by the fibrillar aggregation of alpha-synuclein in either neurons or glial cells. Alpha-synuclein aggregation is potentially causative of disease progression, as variants in alpha-synuclein that promote aggregation are associated with early-age disease onset and familial forms of PD and DLB. In PD, alpha-synuclein aggregation occurs primarily in dopaminergic neurons, while in MSA aggregation is primarily in oligodendrocytes. Natively, alpha-synuclein functions as a vesicle transport protein. αSyn is an intrinsically disordered protein, which has prevented determination of an atomic structure of its soluble form. However, multiple structures of fibrillar alpha-synuclein have been determined, both of recombinant and brain-derived fibrils.

There is a need in the art for compositions having the ability to disaggregate tau and alpha-synuclein fibrils in aqueous conditions into smaller non-toxic species, and methods for making and using such compositions.

### SUMMARY OF THE INVENTION

To illuminate the remarkable but so far unexplained mechanism of EGCG's disaggregation of tau amyloid, we cryogenically trapped an intermediate on the pathway of EGCG-driven disaggregation of tau fibrils extracted from post-mortem brains of AD patients. These studies on the EGCG-AD-tau-fibril complex resulted in the discovery of what we term the “EGCG pharmacophore” on tau fibrils. This EGCG pharmacophore is the ensemble of steric and electronic features on tau fibrils necessary to ensure the optimal supramolecular interactions with EGCG and to trigger EGCG biological effects on tau fibrils.

As discussed below, building upon these discoveries, we used this fibril pharmacophore in a variety of methods such as those designed for an in silico screening of a library of drug-like small molecule compounds with properties predictive of central nervous system penetration. Following this screening methodology, we identified a number of tau-disaggregating molecules with physiochemical drug-like properties superior to those of EGCG. In addition to these screening methods, embodiments of the invention include compositions comprising these tau-disaggregating molecules and methods for making and using them. Tau pathology likely propagates throughout the brain by prion-like seeding, in which aggregates in one diseased cell travel to adjacent cells and induce further protein aggregation ¹⁶⁻¹⁸. Thus, we also characterized the cytotoxicity and prion-like seeding capacity AD-tau fibrils after disaggregation by our lead small molecule disaggregants. Our analysis of EGCG-AD-tau-fibril complex provides evidence for how a small molecule can disassemble stable fibril architecture.

In addition, we have also discovered small molecules capable of disassembling pre-formed alpha-synuclein fibrils. The compounds disclosed herein disaggregate recombinant alpha-synuclein fibrils in vitro, prevent the intracellular seeded aggregation of alpha-synuclein fibrils, and mitigate alpha-synuclein fibril cytotoxicity in neuronal cells. Furthermore, we demonstrate that both compounds disassemble fibrils extracted from MSA patient brains and prevent their intracellular seeding. They also reduce in vivo alpha-synuclein aggregation in C. elegans. Both compounds also penetrate brain tissue in mice. A molecular dynamics-based computational model suggests the compounds may exert their disaggregating effects on the N-terminus of the fibril core. These compounds appear to be therapeutic leads for targeting alpha-synuclein for the treatment of synucleinopathies.

As discussed in the sections below, the invention disclosed herein has a number of embodiments based upon the above-noted discoveries. Embodiments of the invention include pharmaceutical compositions comprising molecules capable of disassembling tau amyloid fibrils or alpha-synuclein amyloid fibrils, as well as methods for making compositions comprising these molecules. Embodiments of the invention also include methods for using these molecules to facilitate the disassembling of tau amyloid fibrils or alpha-synuclein amyloid fibrils.

Embodiments of the invention include, for example compositions of matter comprising at least one of small molecule disaggregant: CNS-2; CNS-11; CNS-11G; CNS-12; CNS-17; MOL01; MOL06; MOL18; or 1541; and a pharmaceutically acceptable carrier. In certain embodiments of the invention, these compositions further comprise amyloid fibrils. For example, certain compositions of the invention include at least one of: CNS-2; CNS-11; CNS-TTG; CNS-12; CNS-17; MOL01; MOL06; MOL18; or 1541; in combination with tau amyloid fibrils or alpha-synuclein amyloid fibrils (e.g., such amyloid fibrils disposed in an in vitro environment). In typical embodiments, the CNS-2; CNS-11; CNS-11G; CNS-12; CNS-17; MOL01; MOL06; MOL18; or 1541 is bound to pharmacophores/sites on tau amyloid fibrils or alpha-synuclein amyloid fibrils.

Embodiments of the invention also include methods of identifying additional agents capable of binding the EGCG pharmacophore of amyloid fibrils. Typically, such methods comprise: disposing fibrils in an aqueous solution in a container; combining the fibrils with one or more test agents; and then observing the ability of the one or more test agents to bind the fibrils; wherein the method includes: observing binding of the one or more agents to very specific locations on fibrils, namely those pharmacophores/sites on fibrils that are bound by epigallocatechin gallate (EGCG), such as the site 1 binding cleft (see, e.g., FIG. 1). In certain embodiments of the invention, such methods include at least one of a cryogenic electron microscopy step; and/or in silico screening of a library of drug-like small molecule compounds; and/or observing the total binding energy of a test agent/fibril pharmacophore complex. In some embodiments, the method includes a step of cryogenically trapping a disaggregant (e.g., EGCG or a small molecule disclosed herein) to amyloid fibrils (e.g., tau fibrils extracted from patients' brains).

Another embodiment of the invention is a method for reducing or inhibiting tau amyloid fibril or alpha-synuclein amyloid fibril aggregation, comprising contacting Tau or alpha-synuclein amyloid protofilaments with an effective amount of one or more of CNS-2; CNS-11; CNS-11G; CNS-12; CNS-17; MOL01; MOL06; MOL18; or 1541 such that tau amyloid fibril or alpha-synuclein amyloid fibril aggregation is inhibited and/or tau amyloid fibril or alpha-synuclein amyloid fibril disaggregation is facilitated. Such methods can be carried out in vitro or in vivo. Another aspect of the invention is a method for restoring the conformation of a Tau or alpha-synuclein protein molecule having an aberrant conformation. An “aberrant conformation,” as used herein, refers to a conformation which is different from the wild type conformation, and which results in a loss of function of the molecule. Such aberrant conformation is sometimes referred to herein as pathological conformation. The aberrant conformation can take the form of amyloid aggregates or fibers (fibrils) of Tau or alpha-synuclein proteins with other molecules or with other proteins. Alternatively, the aberrant conformation can take the form of misfolding (e.g., partial or complete unfolding) of the Tau or alpha-synuclein protein due to mutations or other factors. In this method for restoring the conformation of a Tau or alpha-synuclein protein having an aberrant conformation, the Tau or alpha-synuclein molecule having the aberrant conformation is contacted with an effective amount of CNS-2; CNS-11; CNS-11G; CNS-12; CNS-17; MOL01; MOL06; MOL18; or 1541. The contacted Tau or alpha-synuclein molecule then has a restored conformation, and exhibits a restored or reactivated biological or biochemical activity.

Other objects, features and advantages of the present invention will become apparent to those skilled in the art from the following detailed description. It is to be understood, however, that the detailed description and specific examples, while indicating some embodiments of the present invention, are given by way of illustration and not limitation. Many changes and modifications within the scope of the present invention may be made without departing from the spirit thereof, and the invention includes all such modifications. The sections below disclose and describe further aspects, methods and/or materials in connection with the invention disclosed herein. Certain aspects of the invention are also shown in Seidler et al., Nature Communications volume 13, Article number: 5451 (2022) (hereinafter “Seidler et al.”) and Murray et al., Proceedings of the National Academy of Sciences of the United States of America, 9 Feb. 2023, 120(7):e2217835120 (hereinafter “Murray et al.”), the contents of both of which are incorporated by reference.

### BRIEF DESCRIPTION OF THE DRAWINGS

FIG. 1: CryoEM structure of AD-tau PHF in complex with EGCG. a. Epigallocatechin gallate (EGCG) is the most abundant polyphenol in green tea. It includes a benzenediol ring (A) adjoined to a tetrahydropyran moiety (C), which are connected to a galloyl ring (D) and pyrogallol ring (B). The 8 hydroxyl groups allow EGCG to engage in hydrogen-bonding and other polar interactions with numerous biomolecules. b. Electron micrographs of brain derived PHFs over the course of EGCG incubation. Without EGCG (top), numerous fibrils are observed. After 3-hour incubation at 370 (middle), subtle changes in the fibril morphology are present, with widening of the fibril fuzzy coat. Far fewer fibrils are seen at this time point. After overnight EGCG incubation (bottom), the rare remaining fibrils appear swollen and disturbed. c. Cross-sectional view of the AD patient brain-derived tau PHF cryoEM structure before the addition of EGCG. d. Tau PHF structure following 3-hour incubation with EGCG. Three new regions of density become apparent with the addition of EGCG (Sites 1-3). Site 1 is located in the polar cleft at the intersection of the two protofilaments composing the PHF. Sites 2 and 3 of new density are observed adjacent to K343 and K347 near the β-helix of the fibril. Both Sites 2 and 3 display weaker density than Site 1. e. Tilted view of the 3-hour structure with EGCG bound at Site 1. f. Close up top- and side-views of EGCG in Site 1. This region borders N327, H329, E338, and K340 of the fibril, with EGCG making polar and hydrogen-bond contacts with these residues. EGCG adopts a primarily planar conformation when bound to the fibril, stabilized by pi-pi interactions of the stacked aromatic rings of EGCG. When viewed from the side of the fibril axis, the EGCG density is seen stacking with the same period as the fibril layers. g. Side-view of a single EGCG molecule buried by the fibril and other copies of EGCG.

FIG. 2: In silico and in vitro screening of tau disaggregants using EGCG pharmacophore. a. To identify novel compounds capable of fibril disaggregation, we performed an in-silico screen using the EGCG binding site to the tau PHF (red circle). Two libraries of compounds were docked to the site using two computational methods (AutoDock and Rosetta), and hits were ranked and selected for experimental characterization. b-c. Distribution of in silico docking scores of compound libraries using AutoDock (b) and Rosetta (c). For both methods, more negative scores indicate stronger compound binding. EGCG was a control for each method. As shown, both methods identify EGCG as a strong binder to the site on the tau PHF. d. Top hits from the computational screen were selected for experimental characterization. Compounds were initially screened using an in vitro biosensor cell assay. Brain-derived tau fibrils were incubated with and without inhibitor compound. Fibrils were then dissolved in liposomes and transduced into HEK293T cells overexpressing fluorescently labelled tau. When fibrils are transfected into the cells, the exogenous seeds initiate the aggregation of the endogenous tau, resulting in the formation of intracellular fluorescent puncta. If fibrils are effectively disaggregated by an inhibitor compound, the exogenous fibril seeds will be dissolved, and the intracellular tau will remain soluble, with no puncta formed. e. Quantification of hit compounds in tau biosensor cell assay. For fibrils treated with DMSO vehicle control (turquois bar), many fluorescent aggregates are seen. Without the addition of fibrils, (“no seed”), no intracellular aggregation occurs. Incubation of fibrils with EGCG also prevents the formation of any seeds. Dashed line indicates 50% reduction in number of aggregates. Yellow bars indicate any compound that produces a >50% reduction in aggregate formation. f. Fluorescent microscopy images of biosensor cells without fibril seeds added (top), with seeds and DMSO control (middle), and with seeds and EGCG (bottom). Numerous bright intracellular puncta are seen in the DMSO control, which are eliminated with the addition of EGCG. All error bars represent ±SD, all experiments were performed with n=3 experimental replicates.

FIG. 3: Characterization of tau disaggregation by lead compounds. a. Top hits from the in silico and biosensor cell screens were selected for further experimental characterization. Four compounds were selected, CNS-11, CNS-17, CNS-2, and CNS-12. b. Electron micrographs of brain-derived tau fibrils after incubation with each compound, with EGCG as control. Few fibrils are observed with EGCG treatment, as well as with CNS-11 and CNS-12. Scale bars represent 250 nm. c. Quantitation of fibril number present on EM images with and without compound treatment. N=33 images were taken from random points on the EM grid, and fibrils were counted. A large reduction in visible fibrils is seen for CNS-11. d. Brain derived tau fibrils were treated with compound and the insoluble fraction was analyzed by Western blot, staining for total tau. e. Quantitation of insoluble tau abundance in the Western blot. Similarly, both EGCG and the lead compounds substantially reduce amount of insoluble tau in the fraction. N=3 experimental replicates were performed for each treatment condition. f. MTT cytotoxicity assay in Neuro2a cell model. Brain-derived tau PHFs with and without vehicle control (PBS) show no toxicity (blue bars). Compounds alone show varied toxicities (dark orange bars). Compounds incubated with tau fibrils (light orange bars) do not show additional toxicity. g. Model of CNS-11 docked to the EGCG binding site on the tau PHF. CNS-11 is within hydrogen bonding distance of both H329 and K340. All error bars represent ±SD.

FIG. 4: Structure-informed mechanism for EGCG-driven disaggregation of AD-tau PHF. Solvation energy calculations of tau PHF structures without EGCG (a) and with EGCG after 3-hour incubation. b. Red residues are more stable; blue residues are less stable. The most stable residues seen across both structures are hydrophobic and buried within the fibril core, and less stable residues are typically on the solvent exposed surface. At 3-hours incubation, the structure is less stable (˜28.1 kcal/mol/chain) than without EGCG (˜34.9 kcal/mol/chain). b. To understand the localized effects in fibril stability, energy difference maps were calculated. Subtraction of the no-EGCG model from the 3-hour EGCG model shows a large shift in free energy of Lys340 at the EGCG binding site, indicating the presence of EGCG significantly destabilizes Lys340. d. The 3-hour PHF-EGCG structure reveals EGCG molecules stacked 4.8 A apart, permitting each EGCG molecule to H-bond with individual stacked molecules of tau (dashed yellow lines connecting EGCG to tau side chains Asn327 and His329). The 4.8 A spacing between tau molecules is characteristic of the intermolecular β-sheet hydrogen bonding distance (dashed yellow lines connecting tau molecules). However, this 4.8 A spacing incurs unfavorable voids between EGCG rings A, C, and D (as indicated by gaps between space-filling atoms). e. The voids between stacked aromatic groups can be filled by compressing the distance between these A, C, and D aromatic rings which face the solvent. In so doing, the EGCG stack curves, widening the spacing on the fibril-facing surface. Asn327 and His329 can maintain favorable hydrogen bonding with the curved stack of EGCG molecules only if the tau molecules separate wider than 4.8 A. This separation would allow water to solvate the separated tau molecules. The curvature of the EGCG stack fills the unfavorable voids between EGCG aromatic rings, and further widens the separation between tau molecules. By this mechanism, binding energy between stacked EGCG molecules is converted to a conformational change that pries apart stacked tau molecules. f. Alternate view showing a tau PHF protofilament being disrupted by curvature of stacked EGCG molecules. g. Reaction coordinate diagram describing the possible mechanism of tau disaggregation by EGCG. Tau PHFs in solution with EGCG (coordinate A) are bound by repeating stacks of EGCG molecules (coordinate B). Once EGCG is bound, local charge-mediated effects begin to destabilize the fibril (coordinate C). These effects include unfavorable burying of charged residues (e.g. Lys340), and disruption of pairing between charged side-chains. These repulsive forces, in addition to possible backbone H-bonding between tau and EGCG, weaken the β-sheet H-bond network of the fibril. Lastly, conformational changes induced by EGCG pi-pi stacking (described in d-f) may further disrupt the fibril architecture, leading to the disaggregated EGCG-bound tau end product (coordinate D).

FIG. 5: In vitro characterization of alpha-syniclein fibril disaggregation. a. Thioflavin T fluorescence of alpha-synuclein fibrils incubated with equimolar EGCG, CNS-11, or CNS-11g for 48 h. A reduction in ThT signal is observed for compound treated samples, indicating a reduction in alpha-synuclein fibrils. b. Representative transmission electron micrographs of alpha-synuclein fibrils with CNS-11 and CNS-11g, showing a reduction in fibril count after compound treatment. Scale bars represent 200 nm. c. Quantification of alpha-synuclein fibrils from TEM images after 48 hours compound incubation. N=10 images from random regions of TEM grids were quantified for each treatment condition. d. Western blot of alpha-synuclein fibrils treated with CNS-11 and CNS-11g, staining for alpha-synuclein. A band for monomeric alpha-synuclein can be seen at 14 kDa, and a smear of higher molecular weight species is also observed. The amount of high MW species is reduced after treatment with both compounds e. Chemical structures of CNS-11 and CNS-11g. All error bars represent±SD. (**, p<0.01; ***, p<0.001) using a one-way ANOVA with pairwise t-test).

FIG. 6: Inhibition of intracellular seeding in alpha-synuclein biosensor cells and mitigation of alpha-synuclein cytotoxicity by CNS-11 and CNS-11g. a. Fluorescent microscopy images of alpha-synuclein biosensor cells with and without compound treatment. HEK293T cells expressing YFP-labelled A53T alpha-synuclein, termed biosensor cells, are seeded with exogenous alpha-synuclein fibrils. After seeding, the soluble fluorescent protein is incorporated into intracellular aggregates, visible as bright puncta on fluorescence microscopy (“αSyn fibrils”, white arrows). Without addition of fibril seeds, no fluorescent puncta are observed (“no fibrils”). Incubation of CNS-11, CNS-11g, or EGCG with the exogenous fibrils before seeding results in a reduction of visible puncta. Scale bar represents 50 PM. b-d. Quantification of fluorescent puncta from the biosensor cells. A dose-dependent decrease in puncta with increasing concentrations of compound pre-treatment can be seen for CNS-11 (b), CNS-11g (c), and EGCG (d). “No inhibitor” indicates cells seeded with fibrils not treated with any compound. e-f. MTT toxicity assay of neuronal cells after treatment with alpha-synuclein fibrils with and without inhibitor. N2a cells were treated with fibrillar alpha-synuclein overnight, resulting in a 40-60% reduction in cell viability. CNS-11 was unable to rescue the alpha-synuclein cytotoxicity; however, CNS-11g did show a dose-dependent rescue of cell viability with increasing concentrations of compound. “No fibrils” indicates untreated cells. N=3 experimental replicates were used for each treatment condition. Error bars represent ±SD.

FIG. 7: Effects of CNS-11 and CNS-11g on patient brain-derived alpha-synuclein fibrils. a. TEM images of alpha-synuclein fibrils extracted from brains of patients with multiple system atrophy. b. CNS-11 and CNS-11g were incubated with MSA brain-derived fibrils for 3 days. TEM images of CNS-11 g treated fibrils shows evidence of fibrils actively disaggregating (red arrow: formed fibril; white arrow: disaggregated fibril). c-e. Quantitation of EM images of MSA brain-derived fibrils treated with no compound (c), CNS-11 (d), or CNS-11g (e) over multiple days. N=10-15 images were taken per experimental condition, with each condition performed in triplicate. Average fibril count per image remains relatively stable for the “no inhibitor” control sample, but a reduction of fibrils is seen for both compound treated samples over the course of three days. f. Alpha-synuclein biosensor cells seeded with MSA patient-derived alpha-synuclein fibrils. With the addition of the fibril seeds (“+MSA fibrils”), numerous fluorescent puncta are visible (white arrows). Treatment with compounds EGCG/CNS-11/CNS-11g greatly reduce the number of puncta visible. g-i. Quantification of seeded aggregates from MSA fibril treated biosensor cells. EGCG (g), CNS-11 (h) and CNS-11g (i) all show a robust effect on reducing intracellular seeding when pre-incubated with fibril seeds prior to transduction into the cells. N=3 experimental replicates were analyzed for each treatment condition. Error bars represent z SD.

FIG. 8: In vivo effects of CNS-11 and CNS-11g in C. elegans. a. C. elegans overexpressing CFP- and YFP-fused alpha-synuclein were treated with CNS-11 and CNS-11g at L1 larval stage then imaged by fluorescent microscopy at Day 6 adulthood to assess for alpha-synuclein aggregation. Pseudo-colored images show numerous punctate aggregates of fluorescent alpha-synuclein in the head region of vehicle treated control worms (no inhibitor), and a reduction of aggregates is observed for those treated with either CNS-11 or CNS-11g. b. Quantification of number of aggregates observed in the head region of each treatment group shows a reduction in aggregates for both CNS-11 and CNS-11g treated worms. c. Untreated and compound treated C. elegans were homogenized and Western blot analysis of insoluble alpha-synuclein (found in sample pellet (P)) and soluble alpha-synuclein (found in sample supernatant (S)) was performed. Without treatment, the majority of alpha-synuclein found in the sample homogenate was found in the insoluble (P) fraction. However, with treatment of either compound, particularly CNS-11g, most of the alpha-synuclein is converted to the soluble (S) fraction. R-actin control is shown below. For each treatment condition n=30 worms were analyzed for both the microscopy and Western blot analysis. Error bars represent z SD.

FIG. 9: Brain penetration of CNS-11 and CNS-11g in mice. Mice were injected intravenously with 0.5 mg/kg of CNS-11 or CNS-11g (n=3 per compound) and sacrificed 1 hour after dosing. Compound levels plasma (a) and brain tissue (b) were analyzed using an LC-MS/MRM method.

FIG. 10: Molecular dynamics simulation of CNS-11 and CNS-11g in complex with alpha-synuclein fibril. a. Atomic structure of recombinant alpha-synuclein fibril (PDB code 6cu7). Both CNS-11 and CNS-11g were docked into four potential binding sites along the fibril surface (Site 1-Site 4) using AutoDock Vina. Key residues at each binding site are labeled (color/numbered circles indicate residue site and number). b-c. Molecular dynamics simulations were performed for each compound docked to each of the four unique binding sites of the alpha-synuclein fibril to model fibril disaggregation and intra-strand spacing of the fibril layers was measured at residues within radius of the selected binding site. (b) At the beginning of the simulation, spacing between the top two layers of the fibril structure is ˜4.8 A (red and blue strands), the standard inter-strand distance found in amyloid fibrils (top images, “0 ns”). After 10 ns of simulation, the distance between the top two fibril layers is assessed. For the fibril with no compound bound, the 4.8 A spacing is generally maintained across all simulations. The addition of compound CNS-11 to Site 2 along the fibril results in a separation between strand layers. (c) Measurements of intra-strand spacing for MD simulations of the unbound fibril, or fibril in complex with CNS-11 or CNS-11g performed at the four different compound binding locations. When bound to Site 1, CNS-11 noticeably disrupts the fibril structure, causing an increase in intra-strand spacing. At Site 2, both CNS-11 and CNS-11g lead to fibril disruption, as evidenced by large variations in strand separation over time. When bound to Site 3 or Site 4, very little perturbation is observed for either compound This potentially indicates that the two compounds may be exerting their disaggregating effects at the N-terminus of the fibril core, near Sites 1 and 2, and less likely at the C-terminus near Sites 3 and 4. All MD simulations were performed for a total of 40 ns.

### DETAILED DESCRIPTION OF THE INVENTION

In the description of embodiments, reference may be made to the accompanying figures which form a part hereof, and in which is shown by way of illustration a specific embodiment in which the invention may be practiced. It is to be understood that other embodiments may be utilized, and structural changes may be made without departing from the scope of the present invention. Unless otherwise defined, all terms of art, notations and other scientific terms or terminology used herein are intended to have the meanings commonly understood by those of skill in the art to which this invention pertains. In some cases, terms with commonly understood meanings are defined herein for clarity and/or for ready reference, and the inclusion of such definitions herein should not necessarily be construed to represent a substantial difference over what is generally understood in the art. Many of the aspects of the techniques and procedures described or referenced herein are well understood and commonly employed by those skilled in the art. The following text discusses various embodiments of the invention.

As discussed below, embodiments of the invention include molecules capable of disassembling tau amyloid fibrils or alpha-synuclein amyloid fibrils, as well as methods for making pharmaceutical compositions comprising these molecules. Embodiments of the invention also include methods for using these molecules to inhibit the assembly of and/or facilitate the disassembling tau amyloid fibrils or alpha-synuclein amyloid fibrils. Certain aspects and embodiments of the invention are found in Seidler et al., Nature Communications volume 13, Article number: 5451 (2022) (hereinafter “Seidler et al.”) and Murray et al., Proceedings of the National Academy of Sciences of the United States of America, 9 Feb. 2023, 120(7):e2217835120 (hereinafter “Murray et al.”), the contents of both of which are incorporated by reference.

In Alzheimer's Disease (AD), the disaggregation of tau fibrils may be a therapeutic approach for addressing this pathology. The small molecule EGCG, abundant in green tea, has long been known to disaggregate tau and other amyloid fibrils, but EGCG has poor drug-like properties, failing to fully penetrate the brain. As discussed below, we have cryogenically trapped an intermediate of brain-extracted tau fibrils on the kinetic pathway to EGCG-induced disaggregation and have determined its cryoEM structure. The structure reveals that EGCG molecules stack in polar clefts between the paired helical protofilaments that pathologically define AD. Treating the EGCG binding position as a pharmacophore, we computationally screened thousands of drug-like compounds for compatibility for the pharmacophore, discovering several that experimentally disaggregate brain tau fibrils. This work illustrates the potential of structure-based, small-molecule drug discovery for amyloid diseases.

In addition, the amyloid aggregation of alpha-synuclein within the brain is associated with the pathogenesis of Parkinson's disease (PD) and other related synucleinopathies, including multiple system atrophy (MSA). Alpha-synuclein aggregates are a major therapeutic target for treatment of these diseases. We identify two small molecules capable of disassembling pre-formed alpha-synuclein fibrils. The compounds disaggregate recombinant alpha-synuclein fibrils in vitro, prevent the intracellular seeded aggregation of alpha-synuclein fibrils, and mitigate alpha-synuclein fibril cytotoxicity in neuronal cells. Furthermore, we demonstrate that both compounds disassemble fibrils extracted from MSA patient brains and prevent their intracellular seeding. They also reduce in vivo alpha-synuclein aggregation in C. elegans. Both compounds also penetrate brain tissue in mice. A molecular dynamics-based computational model suggests the compounds may exert their disaggregating effects on the N-terminus of the fibril core. These compounds appear to be therapeutic leads for targeting alpha-synuclein for the treatment of synucleinopathies.

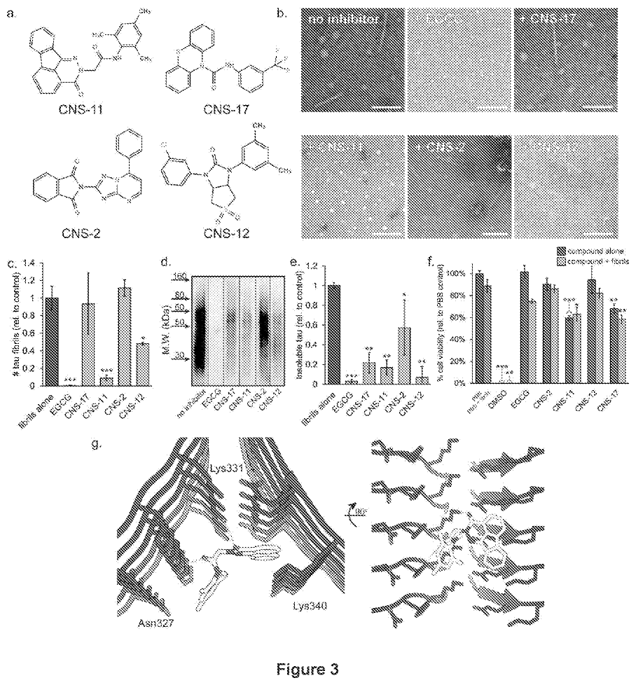

As noted above, a number of molecules have been identified that can facilitate the disaggregation of tau and/or alpha synuclein. The molecules that target tau fibrils include CNS-2; CNS-11; CNS-12; CNS-17; MOL18 and 1541. The molecules that target alpha synuclein fibrils include CNS-11; CNS-11G; MOL01; and MOL06. The chemical formulae of CNS-2; CNS-12; and CNS-17 are shown in FIG. 3A. The chemical formulae of CNS-11; CNS-11G; MOL01; MOL06; MOL18; or 1541 are shown in Table A. The invention disclosed herein has a number of embodiments. Embodiments of the invention include, for example, a composition of matter comprising at least one of: CNS-2; CNS-11; CNS-11G; CNS-12; CNS-17; MOL01; MOL06; MOL18; or 1541 and a pharmaceutically acceptable carrier. In certain embodiments of the invention, these compositions further comprise amyloid fibrils. For example, certain compositions of the invention include at least one of: CNS-2; CNS-11; CNS-11G; CNS-12; CNS-17; MOL01; MOL06; MOL18; or 1541; in combination with tau amyloid fibrils or alpha-synuclein amyloid fibrils (e.g., such amyloid fibrils disposed in an in vitro environment). In certain embodiments, the CNS-2; CNS-11; CNS-11G; CNS-12; CNS-17; MOL01; MOL06; MOL18; or 1541 is bound to tau amyloid fibrils or alpha-synuclein amyloid fibrils.

Embodiments of the invention include methods of making a pharmaceutical composition comprising combining at least one of: CNS-2; CNS-11; CNS-TTG; CNS-12; CNS-17; MOL01; MOL06; MOL18; or 1541 with a pharmaceutically acceptable excipient such that the pharmaceutical composition is made. In some embodiments of the invention, the pharmaceutically acceptable excipient is one selected for its ability to facilitate oral administration of the composition. In other embodiments of the invention, the pharmaceutically acceptable excipient is one selected for its ability to facilitate parenteral administration of the composition. Certain of these methods include combining these molecules with amyloid fibrils (e.g., by disposing amyloid fibrils in the composition). In certain embodiments, the CNS-2; CNS-11; CNS-11G; CNS-12; CNS-17; MOL01; MOL06; MOL18; or 1541 is disposed in an aqueous solution and bound to tau amyloid fibrils or alpha-synuclein amyloid fibrils.

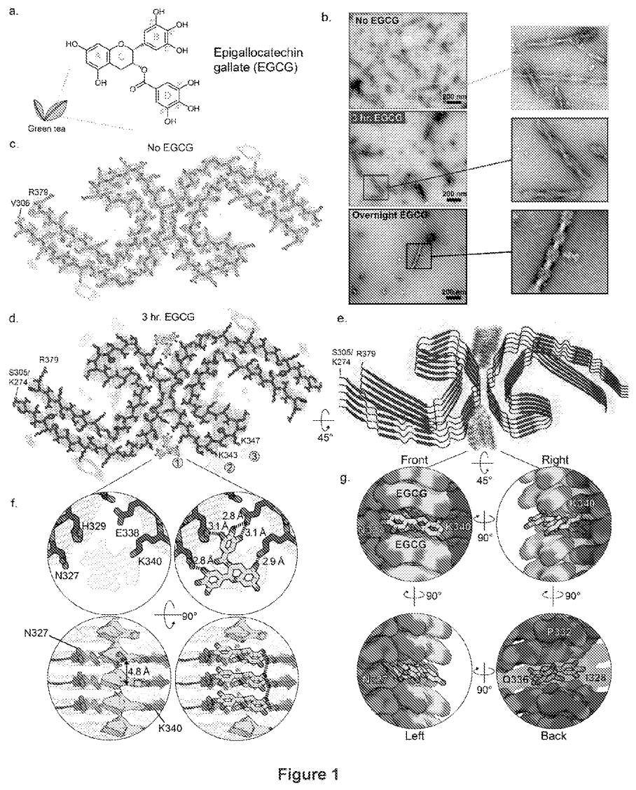

Embodiments of the invention also include methods of identifying agents capable of binding amyloid fibrils as disclosed herein. Embodiments of these methods include assays of new/test molecules in combination with CNS-2; CNS-11; CNS-11G; CNS-12; CNS-17; MOL01; MOL06; MOL18; or 1541 is bound to pharmacophores/sites on tau amyloid fibrils or alpha-synuclein amyloid fibrils that are bound by epigallocatechin gallate (EGCG). Typically, such methods comprise: disposing fibrils in an aqueous solution in a container; combining the fibrils with one or more test agents; and then observing the ability of the one or more test agents to bind the fibrils; wherein the method includes: observing binding of the one or more agents to very specific locations on fibrils, namely those pharmacophores/regions on fibrils that are bound by epigallocatechin gallate (EGCG), such as the site 1 binding cleft (see, e.g., FIG. 1). In certain embodiments of the invention, the method includes at least one of a cryogenic electron microscopy method; and/or in silico screening of a library of drug-like small molecule compounds; and/or observing the total binding energy of a test agent/fibril complex. In some embodiments, the method includes a step of cryogenically trapping a disaggregant (e.g., EGCG or a small molecule disclosed herein) to amyloid fibrils (e.g., tau fibrils extracted from patients' brains).

Another aspect of the invention is a method for reducing or inhibiting tau amyloid fibril or alpha-synuclein amyloid fibril aggregation, comprising contacting Tau or alpha-synuclein amyloid protofilaments with an effective amount of one or more of CNS-2; CNS-11; CNS-11G; CNS-12; CNS-17; MOL01; MOL06; MOL18; or 1541. Such methods can be carried out in vitro (in solution) or in vivo (e.g. in a subject). Another aspect of the invention is a method for restoring the conformation of a Tau or alpha-synuclein protein molecule having an aberrant conformation. An “aberrant conformation,” as used herein, refers to a conformation which is different from the wild type conformation, and which results in a loss of function of the molecule. Such aberrant conformation is sometimes referred to herein as pathological conformation. The aberrant conformation can take the form of amyloid aggregates or fibers (fibrils) of Tau or alpha-synuclein proteins with other molecules or with other proteins. Alternatively, the aberrant conformation can take the form of misfolding (e.g., partial or complete unfolding) of the Tau or alpha-synuclein protein due to mutations or other factors. In this method for restoring the conformation of a Tau or alpha-synuclein protein having an aberrant conformation, the Tau or alpha-synuclein molecule having the aberrant conformation is contacted with an effective amount of CNS-2; CNS-11; CNS-11G; CNS-12; CNS-17; MOL01; MOL06; MOL18; or 1541. The contacted Tau or alpha-synuclein molecule has a restored conformation, and exhibits a restored or reactivated biological or biochemical activity.

A related aspect of the invention is a method for reactivating or restoring a biological or biochemical activity (function) of a Tau or alpha-synuclein protein which results from aberrant conformation of the Tau or alpha-synuclein protein. The method comprises contacting the Tau or alpha-synuclein protein molecule having an aberrant conformation with an effective amount of CNS-2; CNS-11; CNS-11G; CNS-12; CNS-17; MOL01; MOL06; MOL18; or 1541. As a result of contacting the Tau or alpha-synuclein protein having the aberrant conformation, the lost biological or biochemical activity of the Tau or alpha-synuclein molecule is reactivated or restored.

Another aspect of the invention is a method for inhibiting or preventing a loss of a biological or biochemical activity (function), of a Tau or alpha-synuclein protein which results from aberrant conformation of the Tau or alpha-synuclein protein. The method comprises contacting the Tau or alpha-synuclein protein molecule having an aberrant conformation with an effective amount of CNS-2; CNS-11; CNS-11G; CNS-12; CNS-17; MOL01; MOL06; MOL18; or 1541. As a result of contacting the Tau or alpha-synuclein protein having the aberrant conformation, the loss of activity of the Tau or alpha-synuclein molecule is inhibited or prevented.

Another aspect of the invention is a method for treating a subject having a disease or condition which is mediated by loss of function of Tau or alpha-synuclein, such as a pathological syndrome in which Tau has an abnormal conformation (e.g., is aggregated or misfolded). That is, the pathological syndrome is associated with Tau having an aberrant conformation. The method comprises administering to the subject an effective amount of CNS-2; CNS-11; CNS-11G; CNS-12; CNS-17; MOL01; MOL06; MOL18; or 1541. In some embodiments, a cocktail of two or more of these compounds is used.

A “subject” can be any subject (typically a patient) having aggregated (fibrillated) Tau or alpha synuclein molecules associated with a condition or disease which can be treated by a method of the present invention. In one embodiment of the invention, the subject has Alzheimer's disease. In another embodiment of the invention, the subject has Parkinson's disease. Typical subjects include vertebrates, such as mammals, including laboratory animals, dogs, cats, non-human primates and humans.

The CNS-2; CNS-11; CNS-11G; CNS-12; CNS-17; MOL01; MOL06; MOL18; or 1541 aggregation inhibitors of the invention can be formulated as pharmaceutical compositions in a variety of forms adapted to the chosen route of administration, for example, orally, nasally, intraperitoneally, or parenterally, by intravenous, intramuscular, topical or subcutaneous routes, or by injection into tissue. Suitable oral forms for administering the inhibitors include lozenges, troches, tablets, capsules, effervescent tablets, orally disintegrating tablets, floating tablets designed to increase gastric retention times, buccal patches, and sublingual tablets.

The inhibitors of the invention can be systemically administered, e.g., orally, in combination with a pharmaceutically acceptable vehicle such as an inert diluent or an assimilable edible carrier, or by inhalation or insufflation. They can be enclosed in coated or uncoated hard or soft shell gelatin capsules, can be compressed into tablets, or can be incorporated directly with the food of the patient's diet. For oral therapeutic administration, the compounds can be combined with one or more excipients and used in the form of ingestible tablets, buccal tablets, troches, capsules, elixirs, suspensions, syrups, wafers, and the like. For compositions suitable for oral and parenteral etc. administration to humans, the term “excipient” is meant to include, but is not limited to, those ingredients described in Remington: The Science and Practice of Pharmacy, Lippincott Williams & Wilkins, 21st ed. (2006) (hereinafter Remington's).

The inhibitors may also be administered intravenously or intraperitoneally by infusion or injection. Solutions of the inhibitors can be prepared in water, optionally mixed with a nontoxic surfactant. Dispersions can also be prepared in glycerol, liquid polyethylene glycols, triacetin, and mixtures thereof and in oils. Under ordinary conditions of storage and use, these preparations can contain a preservative to prevent the growth of microorganisms.

The pharmaceutical dosage forms suitable for injection or infusion can include sterile aqueous solutions or dispersions or sterile powders comprising the compounds which are adapted for the extemporaneous preparation of sterile injectable or infusible solutions or dispersions, optionally encapsulated in liposomes. In all cases, the ultimate dosage form should be sterile, fluid and stable under the conditions of manufacture and storage. The liquid carrier or vehicle can be a solvent or liquid dispersion medium comprising, for example, water, ethanol, a polyol (for example, glycerol, propylene glycol, liquid polyethylene glycols, and the like), vegetable oils, nontoxic glyceryl esters, and suitable mixtures thereof. The proper fluidity can be maintained, for example, by the formation of liposomes, by the maintenance of the required particle size in the case of dispersions or by the use of surfactants.

Sterile injectable solutions are prepared by incorporating the compounds in the required amount in the appropriate solvent with various of the other ingredients enumerated above, as required, followed by filter sterilization. In the case of sterile powders for the preparation of sterile injectable solutions, the preferred methods of preparation are vacuum drying and freeze drying techniques, which yield a powder of the active ingredient plus any additional desired ingredient present in the previously sterile-filtered solutions.

Useful solid carriers include finely divided solids such as talc, clay, microcrystalline cellulose, silica, alumina and the like. Other solid carriers include conventional nontoxic polymeric nanoparticles or microparticles. Useful liquid carriers include water, alcohols or glycols or water/alcohol/glycol blends, in which the compounds can be dissolved or dispersed at effective levels, optionally with the aid of non-toxic surfactants. Adjuvants such as fragrances and additional antimicrobial agents can be added to optimize the properties for a given use. The resultant liquid compositions can be applied from absorbent pads, used to impregnate bandages and other dressings, or sprayed onto the affected area using pump-type or aerosol sprayers.

Useful dosages of the pharmaceutical compositions of the invention can be determined by comparing their in vitro activity, and in vivo activity in animal models. Methods for the extrapolation of effective dosages in mice, and other animals, to humans are known to the art. Effective dosages and routes of administration of agents of the invention are conventional. The exact amount (effective dose) of the agent will vary from subject to subject, depending on, for example, the species, age, weight and general or clinical condition of the subject, the severity or mechanism of any disorder being treated, the particular agent or vehicle used, the method and scheduling of administration, and the like. A therapeutically effective dose can be determined empirically, by conventional procedures known to those of skill in the art. See, e.g., The Pharmacological Basis of Therapeutics, Goodman and Gilman, eds., Macmillan Publishing Co., New York. For example, an, effective dose can be estimated initially either in cell culture assays or in suitable animal models. The animal model may also be used to determine the appropriate concentration ranges and routes of administration. Such information can then be used to determine useful doses and routes for administration in humans. A therapeutic dose can also be selected by analogy to dosages for comparable therapeutic agents.

The particular mode of administration and the dosage regimen will be selected by the attending clinician, taking into account the particulars of the case (e.g., the subject, the disease, the disease state involved, and whether the treatment is prophylactic). Treatment may involve daily or multi-daily doses of compound(s) over a period of a few days to months, or even years.

Further aspects and embodiments of the invention are disclosed in the following examples.

### Example 1: Structure-Based Discovery of Small Molecules that Disaggregate Tau Fibrils from Alzheimer'S Disease

As discussed below, we have cryogenically trapped an intermediate of brain-extracted tau fibrils on the kinetic pathway to EGCG-induced disaggregation and have determined its cryoEM structure. The structure reveals that EGCG molecules stack in polar clefts between the paired helical protofilaments that pathologically define AD. Treating the EGCG binding position as a pharmacophore, we computationally screened thousands of drug-like compounds for compatibility for the pharmacophore, discovering several that experimentally disaggregate brain tau fibrils. Certain aspects of the invention are also shown in Seidler et al., Nature Communications volume 13, Article number: 5451 (2022) (hereinafter “Seidler et al.”), the contents of which are incorporated by reference.

Structure Determination of Tau Filaments in Complex with EGCG

We surveyed the time course of EGCG-driven tau disaggregation by incubating AD brain-derived tau fibrils with EGCG for 1, 3, 6, and 24 hours at 37°. We monitored the reduction of AD tau aggregates over time by dot blot analysis using the monoclonal antibody GT38, which specifically recognizes AD tau fibrils (Extended Data FIG. 1a as shown in Seidler et al.)¹⁹. We observed a reduction in aggregate level beginning at 1-3 hours, while total hyperphosphorylated tau levels remained the same, as determined by dot blot with antibody AT8 (Extended Data FIG. 1b in Seidler et al.). In agreement with these observations, negative stain electron micrographs of the 3-hour fibrils appear largely intact but somewhat swollen, and at later times the fibrils disappear and are nearly gone after 24 hours of incubation (FIG. 1 b). Thus, intermediates in fibril disassembly appear to be most abundant at time points up to 3 hours, according to two lines of evidence.

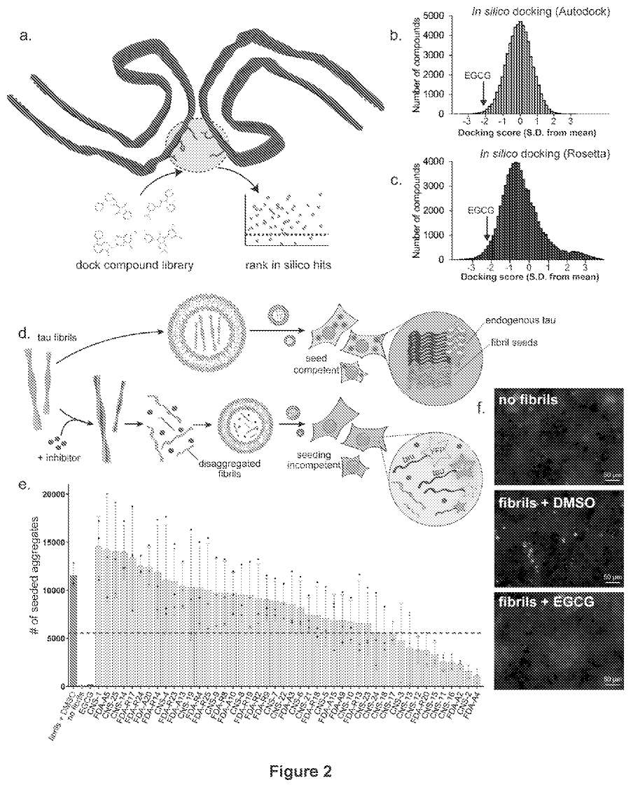

To resolve the tau-EGCG interactions responsible for tau fibril disassembly, we collected and processed cryoEM images at the 1- and 3-hour time points identified above as being enriched in disassembly intermediates (Extended Data FIG. 2 in Seidler et al.). As a negative control, we also collected and processed images of AD tau fibrils before incubation with EGCG (Extended Data FIG. 2 in Seidler et al.). Helical reconstructions of all three datasets revealed the Paired Helical Filament (PHF) tau polymorph, while Straight Filaments, a polymorph of tau fibrils usually found in AD at low abundance, were not present in sufficient quantities for structure determination (FIG. 1 c-d, Extended Data FIG. 3 in Seidler et al.). Extended Data FIG. 3d in Seidler et al. also shows difference maps between the 0 h and 3 h maps.

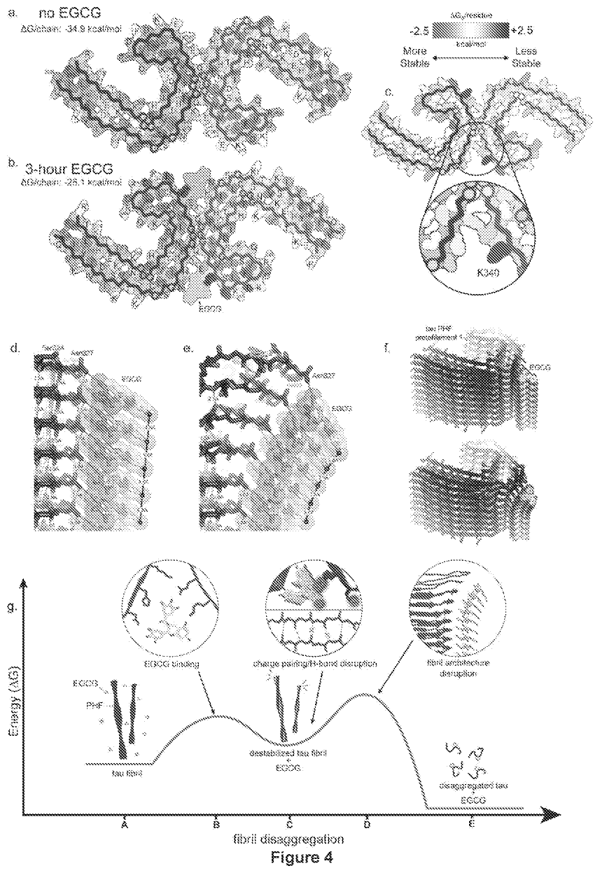

Three new densities alongside the PHF (FIG. 1d; Sites 1-3) were revealed in cryoEM maps of PHFs incubated with EGCG for 1- and 3-hours. These new densities are not present in maps of our control, lacking EGCG (FIG. 1c). Reassuringly, our control map precisely matches previously published tau PHF maps (FIG. 1c)^{20,21}. Moreover, the density attributed to tau exhibits the same backbone path at all time points (FIG. 1 c-d, Extended Data FIG. 3 c-f in Seidler et al.). Consequently, conformational changes among our refined atomic models of the PHFs are minor (Extended Data FIG. 4 a-b in Seidler et al.)^{11,12} and our discussion of EGCG's interaction with tau fibrils focuses on Sites 1-3 in the 3-hour structure, where they appear most strongly.

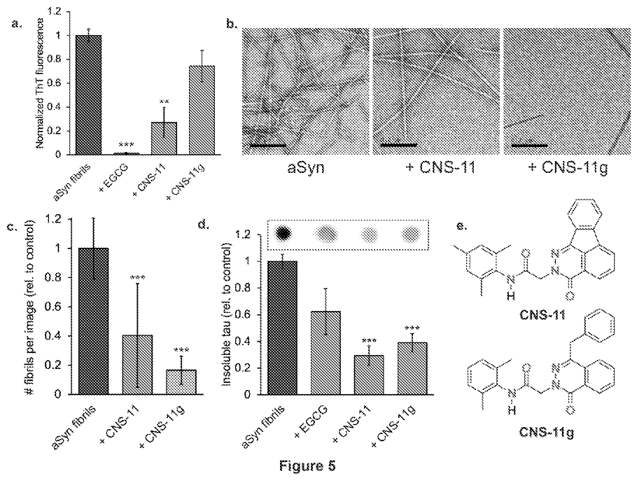

The density at Site 1 is the most prominent of the three sites. It lies in a cleft formed by the junction of two tau protofilaments bordered by the polar residues Asn327, His329, Glu338, and Lys340. It has three distinct lobes, which resemble the three aromatic branches of EGCG, leading us to attribute it to EGCG (FIG. 1d, f). Sites 2 and 3 are located alongside the β-helix region of the fibril core, adjacent to Lys321 and Lys317, respectively. Densities at Sites 2 and 3 are smaller than Site 1 and do not have the characteristic three lobe shape of EGCG. Moreover, an atomic model of EGCG bound to Site 1 buries much greater area on the PHF surface than does EGCG modeled at Sites 2 and 3 (227 Å² vs. 34 Å² and 38 Å², respectively) (Extended Data FIG. 5 in Seidler et al.). Therefore, we focus on Site 1 as the main EGCG pharmacophore.

Our atomic model shows EGCG molecules stacking in two helical columns that span the fibril length: one column at each of the two symmetry-related inter-protofilament clefts. A view perpendicular to the PHF fibril axis shows that Site 1 density repeats every 4.8 A, in register with the spacing between tau molecules (FIG. 1e-g). To model EGCG molecules in such closely spaced densities, we adjusted the EGCG conformation to be nearly planar, thus avoiding steric clash with neighboring EGCG molecules in the stack. EGCG stacking has been observed previously in EGCG crystal form IV²². However, the stacking distance in the crystal structure is nearly 1 Å greater than the 4.8 Å amyloid spacing—a difference accommodated by a greater tilting of the EGCG molecular plane away from the stacking axis' normal plane. It is clear that EGCG complexed with tau does not stack at the crystallographic distance. If that were the case enforcement of helical symmetry during the cryoEM refinement would have produced continuous density across layers. The clean 4.8 A separation of Site 1 densities suggests that EGCG molecules form a 1:1 complex with tau molecules in the fibril.

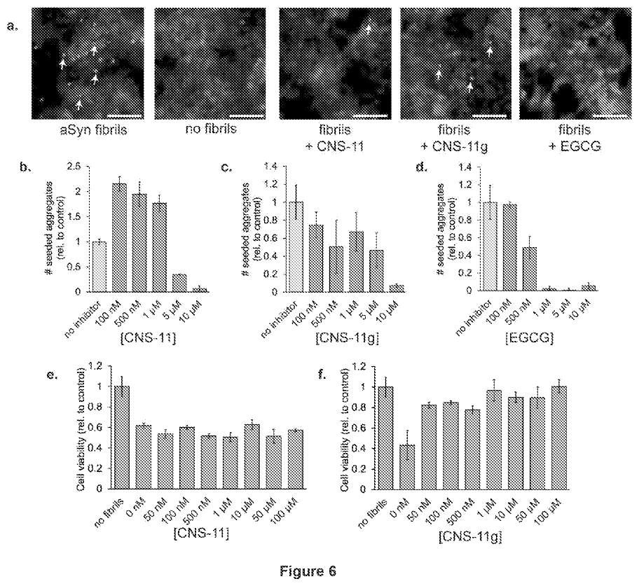

To establish the most likely binding pose of EGCG in the 3-hour map, we assessed 6 different conformations (A-F) using numerous metrics, including the fit of the model to the density, the buried surface area, and the number of hydrogen bonds formed. Conformation C (Extended Data FIG. 6, Table 2 in Seidler et al.) scored moderately better than the others and features multiple stabilizing interactions with the protein side chains. In this pose, the 4′ hydroxyl of the EGCG D-ring hydrogen bonds with His329 and Glu338, and the 3′ and 4′ hydroxyl from the monocyclic B-ring hydrogen bonds with Asn327 (FIG. 1f). A fourth hydrogen bond is made between Lys340 and a hydroxyl from the A ring moiety of EGCG. Partial 7-7 stacking adheres EGCG ring D to the aromatic side chain of His329 (Extended Data FIG. 6c in Seidler et al.).

In summary, EGCG disaggregates AD-tau PHFs over the course of 24 hours, and by structure determination of fibrils after 3 hours of EGCG incubation, we trap an intermediate on the disassembly pathway. This structure reveals EGCG molecules stacked in the two symmetrically related clefts formed at the junction of the two protofilaments. We consider the EGCG binding site on AD-tau fibrils as an EGCG pharmacophore.

### Screening for Tau Disaggregants Using the EGCG Pharmacophore

EGCG itself is a poor therapeutic candidate owing to its polyphenolic molecular structure, which results in unfavorable drug-like properties and restricts brain penetration. Fortunately, our structure provides clues for discovering new disaggregant molecules with more desirable drug characteristics. We hypothesized that effective disaggregants can be identified by computationally selecting molecules complementary in shape to the pharmacophore defined by the trapped EGCG-AD-tau.

For in silico docking we selected two small molecule libraries: (1) currently FDA-approved small molecule drugs (˜1700 compounds), and (2) ChemBridge CNS-set, a ˜60,000 compound library containing drug-like compounds that have characteristics favoring brain-penetration and oral bioavailability (following the Lipinski rule of five, low polar surface area, etc.). EGCG was included as a positive control in both in silico docking libraries. We used two docking methods; AutoDock Vina²³ and RosettaLigand²⁴ to identify compounds that could bind favorably in EGCG binding Site 1 (FIG. 2a). Both docking methods ranked EGCG among the top scoring compounds, with predicted binding energies >2 standard deviations stronger than the average compound (FIG. 2c-b). This result indicates that both docking methods successfully recognize EGCG as a ligand of AD tau fibrils.

From the in silico screen, compounds were ranked and 46 selected for further experimental validation based on total binding energy of the ligand/fibril complex. Experimental assessment consisted of direct measures of fibril disassembly (FIG. 2d-e, Table 3) and assessment of seeding capability of the products of disassembled AD tau fibrils in a biosensor cell assay²⁵. Disaggregating molecules that produce active seeds are undesirable for therapy and are excluded from further study. HEK293T cells expressing CFP- or YFP-fused tau are transduced with exogenous AD tau fibrils. Externally applied AD-tau fibrils then induce the aggregation of the endogenous fluorescent tau, resulting in formation of FRET-positive intracellular aggregates, visible as bright puncta by fluorescence microscopy (FIG. 2f). Automated image analysis of visible puncta provides objective quantification of seeded aggregation. When AD-tau fibrils are pre-treated with compounds capable of disaggregation, the fibrils no longer seed the aggregation of the intracellular tau, and the cells remain diffusely fluorescent, with no visible puncta. Although biosensors may be limited in reporting structural characteristics of fibril progeny²⁶, they offer exquisitely sensitive measures of tau seeding activity—the ability for fibrils to recruit monomeric protein into fibrillar form, which is thought to be the mechanism for tau spreading in AD.

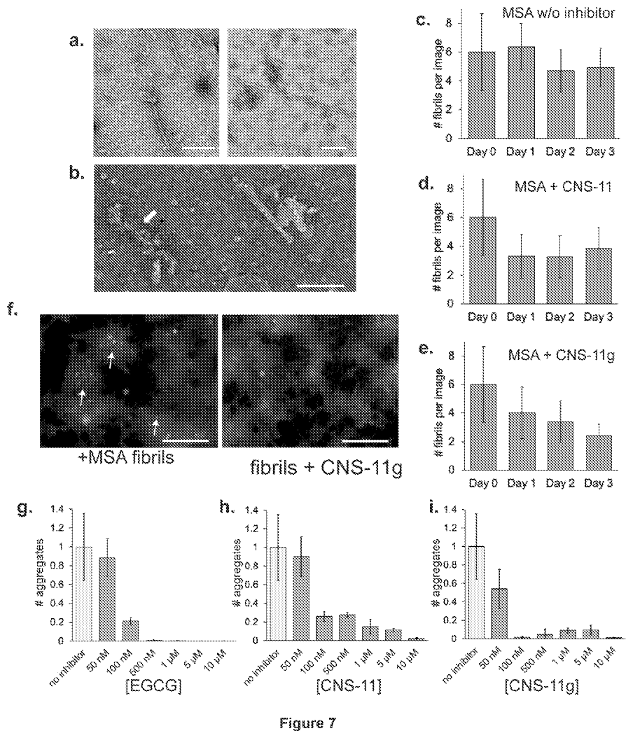

We identified 11 compounds that inhibit the seeding efficiency of crude AD brain extracts by at least 50%; 8 compounds from the CNS-Set library and 3 from the FDA-approved library (FIG. 2e). The top hit, FDA-A4 (Temoporfin), was an effective inhibitor, but was toxic to biosensor cells under the assayed conditions. Activity of FDA-R20 (Phenylbutazone) was modest, so instead we focused our subsequent efforts on FDA-A2 (Lomitapide) and the remaining 8 best CNS-Set compounds by measuring dose-dependent inhibition of seeding by brain-purified AD-tau fibrils. As shown in Extended Data FIG. 7 in Seidler et al., dose-dependent inhibition of seeding is observed for 7 of these 9 compounds.

From the biosensor experiments, 4 of the CNS-Set compounds (CNS-11, 17, 2, and 12) inhibited seeding efficiency with IC₅₀ values <5 μM. These four lead compounds represent a relatively diverse chemical space, with limited similarity between each compound or EGCG (FIG. 3a), and all with favorable drug-like qualities (Table 3). Electron microscopy of tau fibrils treated with each compound reveals a qualitative reduction in fibrils, particularly for CNS-11 and CNS-12 (FIG. 3b). We then scrutinized the four CNS-Set inhibitors by quantitative EM (qEM) imaging (FIG. 3c, see Methods). Inhibitor CNS-11 stands out as having disaggregation activity approaching that of EGCG. CNS-12 also exhibited reduction in AD-tau fibrils, although with lower efficacy. As additional confirmation, we quantified the abundance of insoluble tau after treating AD-tau fibrils with disaggregating compounds using Western blot analysis (FIG. 3d-e). The results mirror qEM data with reductions in insoluble tau by CNS-11 and CNS-12. CNS-17 also showed reduction in insoluble tau species, although corresponding reduction in fibril density was not seen by qEM. The differences may reflect added shear forces that are exerted by SDS-PAGE/Western analysis, which disrupts fibrils that are chemically weakened, but not entirely disaggregated by bound disaggregants. By comparison, qEM is a gentler approach that is more reflective of the isolated effects of disaggregant binding. Lastly, using an MTT assay with Neuro 2a (N2a) cells, we assessed the neuronal cytotoxicity of each compound alone, and after incubation with AD-tau fibrils (FIG. 3f). Apart from CNS-11, no significant cytotoxicity for EGCG or the other lead compounds was detected. Importantly, no significant changes in cytotoxicity are observed for the samples after incubation with AD-tau fibrils, implying the disaggregated fibrils do not gain cytotoxicity.

The reduction of fibrils following EGCG treatment shown by negative-stain EM in FIG. 3b. also rules out a possible alternative explanation for decreased detection of GT38 positive tau aggregation in the dot blot of Extended Data FIG. 1 in Seidler et al.—namely interference of EGCG binding by antibody GT38. That is, the observed reduction of fibrils is produced by the disaggregating action of EGCG, not by interference of EGCG with antibody GT38. Also supporting the action of EGCG as the cause of fibril disaggregation are the data of FIG. 2e, showing EGCG treatment reduces seeding in biosensor cells.

### Molecular Dynamics and Energetics of Compound-Fibril Complexes

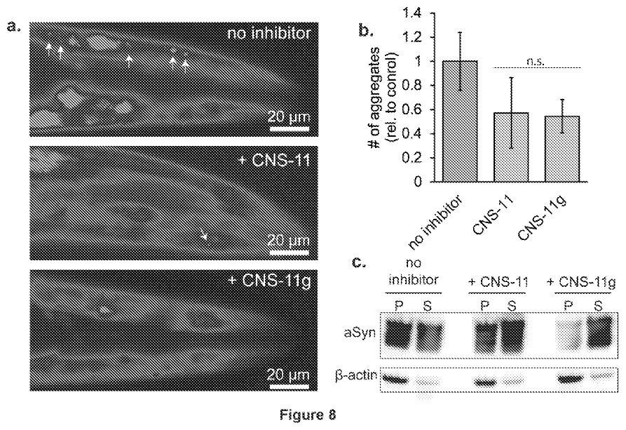

To further understand how bound EGCG and lead compounds from the in-silico screen may be destabilizing the tau PHF structure, we performed molecular dynamics (MD) simulations of each compound/fibril complex. Without a compound bound, the 4.8 Å spacing between layers of the fibril remains stable over the 100 ns duration of MD simulation (Extended Data. FIG. 8a in Seidler et al.). Docking a single EGCG molecule in the Site 1 binding cleft leads to an increase in separation between tau molecules to ˜9 Å, which persists over the duration of MD simulation (Extended Data FIG. 8b in Seidler et al.). Inter-layer spacing was chiefly perturbed by EGCG at the segment spanning residues Lys340-Lys343 of AD-tau, particularly at Glu342 and Lys343. EGCG appears to rapidly form a hydrogen bond with the backbone amide of Ser341, disrupting the hydrogen-bond network between the fibril layers (Extended Data FIG. 8g-h in Seidler et al.). CNS-11 also destabilizes inter-layer spacing by MD, although the major perturbations are centered on Lys340 with additional perturbations seen at Ser341 and Glu342. None of the other CNS-set compounds perturbed the fibril in our simulation, consistent with our experimental results of CNS-11 and EGCG being the most effective disaggregants. These MD experiments suggest that an early contribution to tau fibril disaggregation by effective disaggregants is their competition for hydrogen bonds that bind adjacent tau molecules into tau fibrils.

The role of EGCG in disaggregating AD-tau is further highlighted by calculations of solvation free energies of the unliganded and 3-hour EGCG structures. Our estimates are based on free energies calculated using coordinates of the control and 3-hour structures (FIG. 4a-b). The results show that each tau molecule in the fibril is less stable after 3-hours EGCG incubation (˜28.1 kcal/mol/chain) compared to the no-EGCG structure (˜34.9 kcal/mol/chain). To identify which tau residues are most destabilized by EGCG binding, we subtracted the free energies for each atom of the control structure (more stable) from the 3-hour structure with EGCG present (less stable). The resulting difference energy map reveals 2.5 kcal/mol decreased stability on Lys340, located within the EGCG binding site (FIG. 4c). This decrease is likely due to burial of the positively charged Lys340 by EGCG. Subtle destabilization is evident in other residues lining the EGCG binding cleft (FIG. 4c). Together, these calculations demonstrate that the PHF structure captured after 3 hours of EGCG incubation is overall less stable, with specific residues in the binding cleft contributing most strongly to the destabilization. Further detailed energetic analysis can be found in Supp. FIG. 1.

### Methods

### Preparation of Crude and Purified Brain-Derived Tan Seeds

For purification of paired helical filaments (PHFs) and straight filaments (SFs) from AD brain tissue, extractions were performed according to the previously published protocol without any modifications²¹. Prior to cryoEM grid preparation, AD brain-purified tau fibrils were pre-incubated at 37° C. with 0.5 mM EGCG that was dissolved in PBS in a buffer comprised of 20 mM Tris-HCl pH 7.4, 100 mM NaCl. Control fibrils from the same brain donor were treated identically except for the addition of EGCG. For biosensor seeding assays with crude AD brain extracts, fresh-frozen tissue from neuropathologically confirmed AD cases were thawed and cut into a 0.2-0.3 g sections. Tissue was manually homogenized in a 15 ml disposable tube in 1 ml of 50 mM Tris, pH 7.4 with 150 mM NaCl and then aliquoted to PCR tubes and sonicated in a cuphorn bath for 120 min under 30% power at 4° C. in a recirculating ice water bath, according to reference¹⁷.

### CryoEM Data Collection and Helical Reconstruction

AD brain purified tau fibrils were applied to glow-discharged Quantifoil 1.2/1.3 electron microscope grids (2.6 μl for 1 min), and subsequently plunge-frozen in liquid ethane on a Vitrobot Mark IV (FEI). Data were collected on a Titan Krios (FEI) microscope (operated with 300 kV acceleration voltage and slit width of 20 eV); the 3-hour EGCG pre-incubation dataset was collected with a Gatan Quantum LS/K2 Summit direct electron detection camera. Control and 1-hour EGCG pre-incubation datasets were collected using the Gatan K3 BioQuantum direct electron detection camera. Counting mode movies were obtained with a nominal physical pixel size of 1.07 Å per pixel with a dose per frame 0.63 e-/Å² (K2 data set) with a total of 30 frames at a frame rate of 5 Hz, resulting in a final dose 19 e-/Å² per image. For K3 datasets, the physical pixel size was 0.549 Å per pixel. The dose per frame for the control data set was 1.66 e-/Å² with a total of 48 frames at a frame rate of 20 Hz resulting in a final dose 80 e-/Å² per image, and for the 3-hour EGCG pre-incubation dataset the dose per frame was 1.2 e-/Å² at a frame rate of 20 Hz resulting in a final dose 56 e-/Å² per image. Automated data collection was driven by the Leginon automation software package³⁴.

All datasets were pre-processed using Unblur³⁵ and CTFFIND 4.1.8³⁶. The 3-hour EGCG data set was corrected for magnification anisotropy using Unblur using anisotropy parameters generated from mag_distortion_estimate³⁷ performed on crystalline ice images. Helical tubes were picked manually using e2helixboxer.py³⁸. For the control data set, particles were extracted with a 320-pixel box size and 10% interbox distance. 2D and 3D classification as well as gold-standard refinement were performed using Relion³⁹. A featureless cylinder was used as an initial 3D model. For the 1- and 3-hour data sets, the following data processing strategy was employed. Initial particles were extracted using a 686-pixel box size with a 10% inter-box distance and downscaled by 2. After 2D and 3D classification using a featureless cylinder as an initial model, particles were re-extracted using a 432-pixel box size for the 3-hour data set and 320-pixel box size for the 1-hour data set without downscaling and refined to high resolution using further rounds of 3D classification and gold-standard refinement. The no EGCG and 3-hour reconstructions were sharpened adhoc using RELION postprocess while the 1-hour structure was auto-sharpened using phenix.auto_sharpen⁴⁰.

### Occupancy Analysis

To calculate the occupancy of EGCG density relative to the fibril, we applied three masks over the solvent, over residues 345-352 of the protein backbone, and EGCG Site 1 density. Using the voxel values within the three masks, the EGCG occupancy was calculated according to the following formula:

$$Occupancy = ( max ( EGCG ) - avg ( solvent ) ) / ( max ( 345 - 352 ) - avg ( solvent ) ) .$$

### Atomic Model Building

The highest resolution structure of an AD-brain derived tau PHF (PDB 6HRE) was used as a starting model for atomic model building. 6HRE coordinates were first docked in the 3.8 Å density map of the 3-hour reconstruction as a rigid body, and subsequent manual refinement was performed in COOT⁴¹. EGCG was modeled into the density by first rigid body docking molecule KDH 911 of PDB 4AWM and subsequent real space refinement. Five fibril layers were added according to the symmetry of the helical reconstruction to maintain local contacts between chains in the fibril during structure refinement. We performed automated structure refinement using phenix.real_space_refine⁴². A similar process was performed for the no EGCG control structure, omitting EGCG. The no EGCG fibril structure was rigid body fit into the 1-hour density to achieve the 1-hour structure.

Additional poses of EGCG were considered by manually placing six plausible alternate conformations of EGCG in binding Site 1. After manual and automatic refinement using phenix.real_space_refine, poses were evaluated using: model validation statistics, buried surface area, shape complementarity and free energy⁴³.

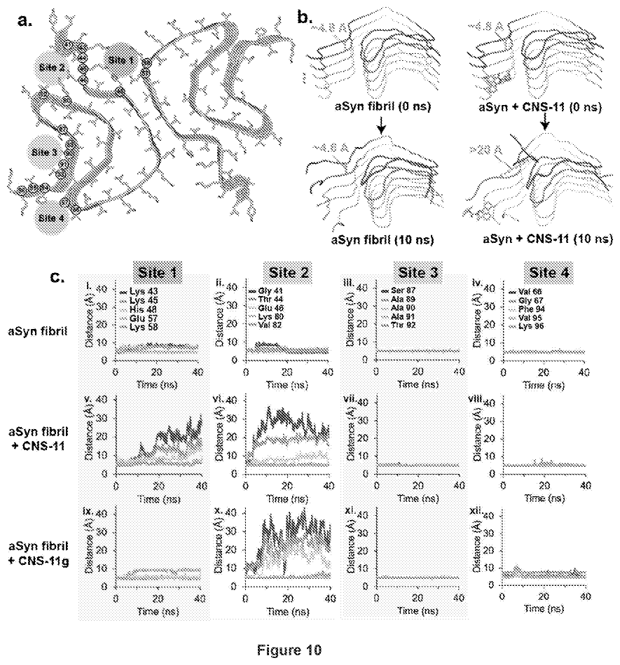

CryoEM data and model refinement statistics are presented in Table 1. Half-map/half-map FSC and model/map FSC curves are shown in Extended Data FIG. 10 in Seidler et al.

### Negative Stain Grid Preparation

Negatively stained EM Grids were prepared by depositing 6 μl of sample on formvar/carbon coated copper grids (400 mesh) for 3 min with. Sample was rapidly wicked using filter paper without drying the grid, and stained with 1% uranyl acetate for 2 min. For quantitative EM image (qEM), Negative-stain EM grids of each sample were screened at a magnification of 11,500×, collecting images in 5-micron increments. Fibrils were counted from collections of 33 micrographs (in triplicate) for each experimental condition.

### Solvation Free Energy Calculations

Solvation free energy calculations were performed as recently described⁴³. Briefly, the solvent accessible surface area (SASA) for each atom on a central strand within an amyloid fibril was determined (folded state). Next, the SASA for each atom of the isolated extended strand was determined (reference state), and the difference between both states was calculated (SASA_{Ref}−SASA_{Fold}). This value was then multiplied by the Atomic Solvation Parameter (ASP) specific to each atom, as determined previously by Eisenberg et al.⁴⁴ An entropic term is also included to take into consideration the degrees of freedom lost in going from a disordered to ordered state⁴⁵. The energies of all atoms were then summed to generate the solvation energy for each structure. Difference energy maps were generated by subtracting solvation free energies pairwise for each atom in the two structures being compared.

### In Silico Docking

Docking calculations were performed using two separate methodologies, AutoDock Vina¹³ and RosettaLigand²⁴. The two compound libraries used for docking were the FDA-approved and CNS-Set from Chembridge. Two-dimensional compound coordinates were downloaded and converted to 3D using Open Babel⁴⁶ For AutoDock Vina, version 1.1.2 was used, and all parameters were kept at default values. A region at the EGCG Site 1 of the tau PHF was defined as the docking site using a 20 Å x16 Å x12 Å box. Compounds were ranked by binding energy. For RosettaLigand, once three dimensional ligand structures were generated using Open Babel, we generated a ligand perturbation ensemble using our previously described method⁴⁷. For each rotatable bond of the ligand a torsion angle deviation of ±5° was applied, generating 100 conformations for each ligand. Ligand docking was performed using the HighResDocker mover in the Rosetta Scripts modality (Rosetta version 3.10), using the Ref2015 energy function. A 7 Å box centered at the EGCG Site 1 binding site on the tau PHF was used as the docking site. Cycles of side chain repacking were coupled with small ligand perturbations every third cycle. Ligand poses were ranked based on lowest binding energy, and interface energies were calculated using the InterfaceScoreCalculator mover.

### Molecular Dynamics Simulations

MD simulations were performed using GROMACS version 2018⁴⁸ and the CHARMM27⁴⁹ all-atom forcefield. The 3-hour tau fibril EGCG complex structure with 10 protein monomers was solvated in a cubic water box using periodic boundary conditions with counter ions added. Systems were energy minimized then temperature and pressure equilibrated for 100 ps. Production runs were carried out for 100 ns. Calculations of non-bonded interactions were gpu accelerated. Calculations were performed with and without bound small molecules. Small molecules were placed into the EGCG Site 1 binding site, with their initial conformations determined by the output from the AutoDock Vina binding (see above). Ligand topologies were calculated using the CHARMM General Force Field server (CGenFF), and hydrogens were added to ligands using Avagadro.

### Inhibitor Screening in Tau Biosensor Cells

HEK293 cell lines stably expressing tau-K18 P301S-eYFP were obtained from Marc Diamond²⁵ and used without further characterization or authentication. Cells were maintained in DMEM (Life Technologies, cat. 11965092) supplemented with 10% (vol/vol) FBS (Life Technologies, cat. A3160401), 1% penicillin/streptomycin (Life Technologies, cat. 15140122), and 1% Glutamax (Life Technologies, cat. 35050061) at 37° C., 5% CO2 in a humidified incubator. Fibrils and patient-derived crude brain extracts were incubated for 16-18 hours at 4° C. with indicated inhibitor to yield a final inhibitor concentration of 10 M (on the biosensor cells), except for IC50 determinations, which instead used adjustments to achieve the final indicated inhibitor concentration. Inhibitors were dissolved in DMSO. For seeding, inhibitor-treated seeds were sonicated in a cuphorn water bath for 3 minutes, and then mixed with 1 volume of Lipofectamine 3000 (Life Technologies, cat. 11668027) prepared by diluting 1 μl of Lipofectamine in 19 μl of OptiMEM. After twenty minutes, 10 μl of fibrils were added to 90 μl of tau biosensor cells. The number of seeded aggregates was determined by imaging the entire well of a 96-well plate in triplicate using a Celigo Image Cytometer (Nexcelom) in the YFP channel. The number of aggregates in a given image were determined using an ImageJ⁵⁰ script, which subtracts the background fluorescence from unseeded cells, and then counts the number of aggregates as peaks with fluorescence above background using the built-in Particle Analyzer. The number of aggregates was normalized to the confluence of each well, and dose-response plots were generated by calculating the average and standard deviations from triplicate measurements.

### MTT Cytotoxicity Assay

90 μL of Neuro2a cells were plated on clear 96 well plates (Falcon 353072) at 6000 cells per well and given 24 hours to adhere to the plate. Subsequent treatment consisted of PHF samples that were coincubated with or without 100 μM of small molecules for 48 hours at 37° C., with 10 μL applied per well for a final small molecule concentration of 10 μM. After another 24-hour incubation at 37° C., 20 μL of Thiazolyl Blue Tetrazolium Bromide MTT dye (Sigma; 5 mg/mL stock in DPBS) was added to each well and further incubated for 3.5 hours at 37° C. Assay was arrested by replacement of all media with 100% DMSO and transfer to a SpectraMax MF reader. Background 700 nm readings were subtracted from the absorbance readings at 570 nm. Cells treated with 100% DMSO were designated as 0% viable and those treated only with vehicle were designated as 100% viable. Other well readings were normalized to these values.

### Western Blot Analysis

AD brain derived tau fibrils were incubated with compounds for 72 hours at 37° C. in PBS. Compounds were centrifuged at 15,000 rpm for 60 minutes to separate soluble and insoluble fractions. Samples were loaded onto gels (NuPAGE 12% Bis-Tris pre-cast) and ran at 200V for 40 minutes. Proteins were transferred from gel to nitrocellulose membrane using an iBLOT2 system. Membrane was blocked for 1 hour in TBST with 5% milk and washed with TBST three times. Membrane was then incubated with primary antibody (anti-tau A0024 (Dako), 1:4000 dilution in 2% milk/TBST solution) for 1 hour, washed three times with TBST, incubated with horseradish peroxidase-conjugated secondary antibody (goat anti-mouse IgG H and L (HRP); 1:5000 Dilution in 2% milk/TBST), and washed three times in TBST. Signal was detected with Pierce ECL Plus Western Blotting Substrate (Cat #32132), and blot was imaged with a Pharos FX Plus Molecular Imager.

### Dot Blot Analysis

Brain-derived tau PHFs were incubated with 80 μM EGCG at 37° C. in PBS various lengths of time. Samples were spotted onto nitrocellulose membrane, 20 μL were spotted for each condition, spotting 2 μL at a time and allowing to dry in between. Membrane was blocked for 1 hour in TBST with 5% milk and washed with TBST three times. Membrane was then incubated with primary antibody (either anti-tau GT38 (obtained from Virginia Lee lab at UPenn) or anti-phospho-tau AT8 (cat #MN1020, lot #UL2906281Z), 1:4000 dilution in 2% milk/TBST solution) for 1 hour, washed three times with TBST, incubated with horseradish peroxidase-conjugated secondary antibody (goat anti-mouse IgG H and L (HRP); 1:5000 Dilution in 2% milk/TBST), and washed three times in TBST. Signal was detected with Pierce ECL Plus Western Blotting Substrate (cat #32132), and blot was imaged with a Pharos FX Plus Molecular Imager.

### DISCUSSION

Our cryogenically trapped structures of AD brain-extracted tau fibrils raise two questions related to the development of drugs for AD. The first is whether the tried-and-true structure-based methods of small-molecule drug discovery that have accelerated treatments for cancer and metabolic diseases can be effectively applied to Alzheimer's disease. The petty pace of drug development for Alzheimer's disease is rooted in part in the stable, traditionally intractable targets, amyloid fibrils. Amyloid fibrils lack the obvious concave active sites of enzymes and mobile helix interaction sites of GPCRs that offer binding cavities for drugs. Also frustrating progress has been the paucity of high-resolution amyloid structures so helpful for drug discovery.

Whereas our structure of 3-hour EGCG-treated AD tau fibrils successfully identifies the useful EGCG pharmacophore, its 3.8 Å resolution falls short of atomic resolution, for several reasons. Chief among these is that we capture the EGCG pharmacophore during the kinetic course of its destruction. We may be averaging tau-EGCG particles that are at different stages of disassembly, leading to blurring in the final reconstruction. The consequent disorder hinders the cryoEM image alignment process. In fact, we were able to determine the less disturbed control and 1-hour samples to 3.4 and 3.3 Å resolutions. Additionally, the occupancy of EGCG at Site 1 is only 66%, indicating that some fibril particles lack EGCG or are at less than full occupancy. This finding is similar to a recent study of PET ligands bound to AD tau having occupancies ranging from ˜40-70%²⁷. Yet despite limited resolution, the wider implication of the EGCG-AD-tau structure is that structure-based drug discovery is possible for amyloid diseases, as it has been for other medical conditions.

In order for structure-based drug discovery efforts for AD to be as successful as efforts in other disease, disaggregants identified as drug leads must be free of toxicity at effective concentrations and must produce products of disaggregation that are not toxic and which do not seed monomeric tau into amyloid fibrils. The natural cellular machinery evolved to disassemble aggregated proteins, including the chaperone²⁸ and ubiquitin ligase systems²⁹. Aids are needed since these systems are quickly overrun as aggregate burden increases and may also exacerbate tau aggregation in the process^{30,31}. We use tau biosensor seeding as a screening assay to identify compounds that produce non-seeding competent products. We note this assay is not definitive, because presently available tau biosensor cells, although sensitive to seeding by a range of tau structures, produce tau aggregates that probably differ in polymorphic form from AD-tau. Absolute assurance that the disaggregated products of AD-tau are not seeding competent in brain awaits further experimentation.

The second major question raised by our structures is how a small molecule can dismantle extraordinarily stable pathogenic amyloid fibrils. Our structure of EGCG bound to AD-tau suggests two possible mechanisms which may operate in concert. The first we label the charge-pairing mechanism. It is based on favorable EGCG-tau interactions that destabilize stacked tau molecules. The second we label the EGCG curvature mechanism and is based on favorable EGCG-EGCG interactions that pry apart the fibril.

Charge-pairing is a well-known stabilizing feature of amyloid fibrils, and its disruption can lead to charge repulsion by like charges stacked in adjacent fibril layers³². Examination of the 3-hour structure shows that EGCG H-bonds to sidechains of tau in the two clefts (Supp. FIG. 2). This can block Lys340 from charge pairing with neighboring Glu338 and Glu342. Once the positive charge of Lys340 no longer compensates the negative charges on the Glu residues, the Glu residues will repel the Glu residues in adjacent layers of tau molecules, weakening the fibril. Our energy difference maps support that EGCG destabilizes the fibril as they show a large increase in energy of Lys340 and the surrounding residues of the EGCG pharmacophore. Tau destabilization may also occur at EGCG Sites 2 and 3, as both sites are adjacent to complementary lysine/aspartic acid charge pairs that may be similarly disrupted by EGCG binding. The disruption of charge pairing may be an early stage of fibril disassembly as it creates repulsion between layers of the fibril, potentially setting the stage for tau molecules to begin separating. As adjacent tau molecules begin to separate, our MD simulations demonstrate that destabilization between tau molecules is energetically compensated by hydrogen bonds between EGCG and the backbone amide of Ser341 (Extended Data FIG. 8 in Seidler et al.). One of the most effective EGCG analogs that we discovered in our screen, CNS-11, is calculated to interact with AD-tau by many of the same hydrogen-bonding interactions as EGCG, including potential interactions with Lys340 and Glu342 (FIG. 3g).

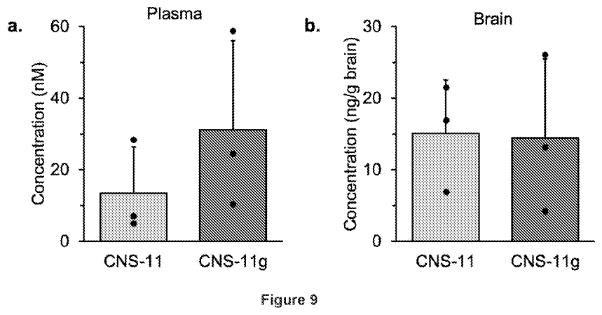

In our second proposed mechanism, interactions between stacked EGCG molecules destabilize fibril integrity. Our structure of EGCG bound to AD-tau reveals EGCG molecules stacked 4.8 Å apart, which permits each EGCG molecule to hydrogen-bond with individual stacked molecules of tau (FIG. 4d). However, this 4.8 Å spacing incurs unfavorable voids between EGCG rings A, C, and D of neighboring molecules in the stacks (gaps between space-filling atoms in FIG. 4d). The stack of EGCG molecules can be stabilized by compressing the distance between the solvent-facing aromatic rings to about 3.5 Å, which achieves van der Waals contact. This compression incurs curvature of the stack and widens the spacing on the fibril-facing surface (FIG. 4e). This curving effect is also observed in an MD simulation of a 5-layer stack of EGCG molecules (Extended Data FIG. 9 in Seidler et al.), and is reminiscent of the tilted interaction between EGCG molecules in the form IV EGCG crystal structure²². Asn327 and His329 can maintain favorable interactions with the curved stack of EGCG molecules only if the tau molecules separate. This separation could allow water to solvate the separated tau molecules and enable other EGCG molecules to invade as seen in our MD simulation. By this mechanism, binding energy between stacked EGCG molecules is converted to a conformational change that pries apart stacked tau molecules (FIG. 4f).

The molecular events described by these two disaggregation mechanisms can be unified in a reaction coordinate diagram (FIG. 4g). First, EGCG binds to the fibril, incurring entropy loss due to its ordering along the fibril surface (FIG. 4g, step B). Next, a quasi-stable intermediate state evolves, represented by the 3-hour structure (FIG. 4g, step C). The intermediate is stabilized by stacking interactions between EGCG molecules and hydrogen bonds between EGCG and the binding cleft. Importantly, the energy well is not inescapably deep, owing to destabilization incurred by EGCG disruption of charge-pairing and the trapping of small voids created between stacked EGCG molecules. Next, tau molecules are pried apart by charge repulsion incurred by EGCG, and curvature induced by elimination of voids between stacked EGCG molecules (FIG. 4g, step D). Lastly, solvent and additional EGCG molecules invade the gaps between tau molecules, completing the disaggregation of tau (FIG. 4g, step E). Thus, our structure offers insight into how a small molecule can dismantle an extraordinarily stable pathogenic amyloid fibril.

We note that the kinetics of disaggregation are relatively slow, particularly for a process that involves high affinity binding of EGCG and breaking of hydrogen bonds. We attribute slow kinetics to our proposed mechanism of disaggregation (FIG. 4d), in which cumulative binding of EGCG causes a concerted conformation change in stacked columns of EGCG, forcing tau layers apart. We suggest this process of conformation equilibrium where forces required to remodel the fibril are counterbalanced by the physiochemical properties of EGCG is a kinetically slow process.

Our proposed mechanism of EGCG mediated AD tau fibril disaggregation is not necessarily specific to tau fibrils. EGCG is known to disassemble fibrils composed of many different amyloid proteins^{7,11}; its effects are not sequence specific, but are instead specific to the amyloid scaffold itself. The hydroxyl-rich EGCG molecule likely seeks out a charge-rich region of any given amyloid fibril, burying charge and stacking planarly alongside it to produce a conformational stress that eventually disassembles it. The promiscuity of EGCG highlights the need in the future to not only identify analogs with better drug-like properties, as we have focused on here, but also to make analogs that are amyloid-specific in order to avoid disaggregating amyloids that form as part of normal biological processes³³.

Here we have determined the intermediate structure of AD tau fibrils on the pathway to EGCG-driven disaggregation. Considering the EGCG binding site a pharmacophore for disaggregating compounds, we identified several tau-disaggregating molecules with physiochemical drug-like properties superior to those of EGCG. Further screening or optimization of these compounds may result in a new generation of AD drug leads capable of entering neurons and effectively disaggregating tau fibrils into inert products. Our approach is broadly applicable to amyloid proteins involved in other amyloid-based degenerative conditions such as Parkinson's disease and systemic amyloidoses.

### Example 1 References

1. La Joie, R. et al. Prospective longitudinal atrophy in Alzheimer's disease correlates with the intensity and topography of baseline tau-PET. Sci. Transl. Med. 12, eaau5732 (2020).
2. Chang, C.-W., Shao, E. & Mucke, L. Tau: Enabler of diverse brain disorders and target of rapidly evolving therapeutic strategies. Science 371, eabb8255 (2021).
3. Soeda, Y. & Takashima, A. New Insights Into Drug Discovery Targeting Tau Protein. Front. Mol. Neurosci. 13, (2020).
4. Congdon, E. E. & Sigurdsson, E. M. Tau-targeting therapies for Alzheimer disease. Nat. Rev. Neurol. 14, 399-415 (2018).
5. LMTM ALZFORUM. https://www.alzforum.org/therapeutics/lmtm.
6. Nagle, D. G., Ferreira, D. & Zhou, Y.-D. Epigallocatechin-3-gallate (EGCG): Chemical and biomedical perspectives. Phytochemistry 67, 1849-1855 (2006).
7. Andrich, K. & Bieschke, J. The Effect of (−)-Epigallo-catechin-(3)-gallate on Amyloidogenic Proteins Suggests a Common Mechanism. Adv. Exp. Med. Biol. 863, 139-161 (2015).
8. Sonawane, S. K. & Chinnathambi, S. Epigallocatechin-3-gallate modulates Tau Post-translational modifications and cytoskeletal network. Oncotarget 12, 1083-1099 (2021).
9. Sonawane, S. K. et al. EGCG impedes human Tau aggregation and interacts with Tau. Sci. Rep. 10, 12579 (2020).
10. Hj, W., A, S., Mi, D., Ee, W. & J, B. The green tea polyphenol (−)-epigallocatechin gallate prevents the aggregation of tau protein into toxic oligomers at substoichiometric ratios. FEBS Lett. 589, (2015).
11. Bieschke, J. et al. EGCG remodels mature α-synuclein and amyloid-D fibrils and reduces cellular toxicity. Proc. Natl. Acad. Sci. 107, 7710-7715 (2010).
12. Chesser, A. S., Ganeshan, V., Yang, J. & Johnson, G. V. W. Epigallocatechin-3-gallate enhances clearance of phosphorylated tau in primary neurons. Nutr. Neurosci. 19, 21-31 (2016).
13. Kocisko, D. A. et al. New Inhibitors of Scrapie-Associated Prion Protein Formation in a Library of 2,000 Drugs and Natural Products. J Virol. 77, 10288 (2003).
14. Roberts, B. E. et al. A synergistic small-molecule combination directly eradicates diverse prion strain structures. Nat. Chem. Biol. 5, 936-946 (2009).
15. Femindez, V. A. et al. Bioavailability of Epigallocatechin Gallate Administered with Different Nutritional Strategies in Healthy Volunteers. Antioxidants 9, (2020).
16. Clavaguera, F. et al. Transmission and spreading of tauopathy in transgenic mouse brain. Nat. Cell Biol. 11, 909-913 (2009).
17. Kaufman, S. K., Del Tredici, K., Thomas, T. L., Braak, H. & Diamond, M. I. Tau seeding activity begins in the transentorhinal/entorhinal regions and anticipates phospho-tau pathology in Alzheimer's disease and PART. Acta Neuropathol. (Berl.) 136, 57-67 (2018).
18. DeVos, S. L. et al. Synaptic Tau Seeding Precedes Tau Pathology in Human Alzheimer's Disease Brain. Front. Neurosci. 12, (2018).
19. Gibbons, G. S. et al. Detection of Alzheimer Disease (AD)-Specific Tau Pathology in AD and NonAD Tauopathies by Immunohistochemistry With Novel Conformation-Selective Tau Antibodies. J. Neuropathol. Exp. Neurol. 77, 216-228 (2018).
20. Fitzpatrick, A. W. P. et al. Cryo-EM structures of tau filaments from Alzheimer's disease. Nature 547, 185-190 (2017).
21. Falcon, B. et al. Tau filaments from multiple cases of sporadic and inherited Alzheimer's disease adopt a common fold. Acta Neuropathol. (Berl.) 136, 699-708 (2018).
22. Smith, A. J. et al. Crystal Engineering of Green Tea Epigallocatechin-3-gallate (EGCg) Cocrystals and Pharmacokinetic Modulation in Rats. Mol. Pharm. 10, 2948-2961 (2013).
23. Trott, O. & Olson, A. J. AutoDock Vina: improving the speed and accuracy of docking with a new scoring function, efficient optimization and multithreading. J. Comput. Chem. 31, 455 (2010).
24. Meiler, J. & Baker, D. ROSETTALIGAND: protein-small molecule docking with full side-chain flexibility. Proteins 65, 538-548 (2006).
25. Holmes, B. B. et al. Proteopathic tau seeding predicts tauopathy in vivo. Proc. Natl. Acad. Sci. 111, E4376-E4385 (2014).
26. Kaniyappan, S. et al. FRET-based Tau seeding assay does not represent prion-like templated assembly of Tau filaments. Mol. Neurodegener. 15, 39 (2020).
27. Shi, Y. et al. Cryo-EM structures of tau filaments from Alzheimer's disease with PET ligand APN-1607. Acta Neuropathol. (Berl) 141, 697-708 (2021).
28. Lindberg, I. et al. Chaperones in Neurodegeneration. J Neurosci. Off J Soc. Neurosci. 35, 13853-13859 (2015).
29. Schmidt, M. F., Gan, Z. Y., Komander, D. & Dewson, G. Ubiquitin signalling in neurodegeneration: mechanisms and therapeutic opportunities. Cell Death Differ. 28, 570-590 (2021).
30. Nachman, E. et al. Disassembly of Tau fibrils by the human Hsp70 disaggregation machinery generates small seeding-competent species. J. Biol. Chein. 295, 9676 (2020).
31. Saha, I. et al. The AAA+ chaperone VCP disaggregates Tau fibrils and generates aggregate seeds. 2022.02.18.481043 Preprint at https://doi.org/10.1101/2022.02.18.481043 (2022).
32. Falcon, B. et al. Novel tau filament fold in chronic traumatic encephalopathy encloses hydrophobic molecules. Nature 568, 420-423 (2019).
33. Fowler, D. M., Koulov, A. V., Balch, W. E. & Kelly, J. W. Functional amyloid—from bacteria to humans. Trends Biochem. Sci. 32, 217-224 (2007).
34. Cheng, A. et al. Leginon: New features and applications. Protein Sci. Publ. Protein Soc. 30, 136-150 (2021).
35. Grant, T. & Grigorieff, N. Measuring the optimal exposure for single particle cryo-EM using a 2.6 Å reconstruction of rotavirus VP6. eLife 4, e06980 (2015).
36. Rohou, A. & Grigorieff, N. CTFFIND4: Fast and accurate defocus estimation from electron micrographs. J Struct. Biol. 192, 216-221 (2015).
37. Grant, T. & Grigorieff, N. Automatic estimation and correction of anisotropic magnification distortion in electron microscopes. J. Struct. Biol. 192, 204-208 (2015).
38. Tang, G. et al. EMAN2: an extensible image processing suite for electron microscopy. J. Struct. Biol. 157, 38-46 (2007).
39. Scheres, S. H. W. RELION: implementation of a Bayesian approach to cryo-EM structure determination. J. Struct. Biol. 180, 519-530 (2012).
40. Terwilliger, T. C., Sobolev, O. V., Afonine, P. V. & Adams, P. D. Automated map sharpening by maximization of detail and connectivity. Acta Crystallogr. Sect. Struct. Biol. 74, 545-559 (2018).
41. Emsley, P., Lohkamp, B., Scott, W. G. & Cowtan, K. Features and development of Coot. Acta Crystallogr. D Biol. Crystallogr. 66, 486-501 (2010).
42. Afonine, P. V. et al. Real-space refinement in PHENIX for cryo-EM and crystallography. Acta Crystallogr. Sect. Struct. Biol. 74, 531-544 (2018).
43. Sawaya, M. R., Hughes, M. P., Rodriguez, J. A., Riek, R. & Eisenberg, D. S. The expanding amyloid family: Structure, stability, function, and pathogenesis. Cell 184, 4857-4873 (2021).
44. Eisenberg, D., Wesson, M. & Yamashita, M. Interpretation of protein folding and binding with atomic solvation parameters. Chem. Scr. 29, 217-222 (1989).
45. Koehl, P. & Delarue, M. Application of a self-consistent mean field theory to predict protein side-chains conformation and estimate their conformational entropy. J Mol. Biol. 239, 249-275 (1994).
46. O'Boyle, N. M. et al. Open Babel: An open chemical toolbox. J. Cheminformatics 3, 33 (2011).
47. Jiang, L. et al. Structure-based discovery of fiber-binding compounds that reduce the cytotoxicity of amyloid beta. eLife 2, (2013).
48. Van Der Spoel, D. et al. GROMACS: fast, flexible, and free. J Comput. Chem. 26, 1701-1718 (2005).
49. Vanommeslaeghe, K. et al. CHARMM general force field: A force field for drug-like molecules compatible with the CHARMM all-atom additive biological force fields. J Comput. Chem. 31, 671-690 (2010).
50. Schneider, C. A., Rasband, W. S. & Eliceiri, K. W. NIH Image to ImageJ: 25 years of image analysis. Nat. Methods 9, 671-675 (2012).

### Example 2: Small Molecules Disaggregate Alpha-Synuclein and Prevent Seeding from Patient Brain Derived Fibrils

Certain aspects of the invention are also shown in Murray et al., Proceedings of the National Academy of Sciences of the United States of America, 9 Feb. 2023, 120(7):e2217835120 (hereinafter “Murray et al.”), the contents of which are incorporated by reference.

The flavonoid epigallocatechin gallate (EGCG) has been previously demonstrated to disaggregate preformed amyloid fibrils^{17,18}. In our previous work, we determined the structure of EGCG in complex with tau paired helical filaments (PHFs) extracted from Alzheimer's disease patient brains¹⁹. From this structure we identified the pharmacophore of EGCG to the tau fibril and proposed its possible mechanism of disaggregation. Using the EGCG pharmacophore as a docking site, we computationally screened a library of ˜60,000 small molecules predicted to have favorable central nervous system penetration based on their biophysical properties (i.e., polar surface area, number of rotatable bonds, etc.). From this screen we identified compounds able to disassemble tau PHFs (in Review), among them the compound we term CNS-11. The ability of EGCG to disaggregate amyloid fibrils is non-specific, as the compound can act upon multiple different amyloid proteins, including tau, alpha-synuclein, and amyloid-beta¹⁷. Thus, we sought to determine if CNS-11 specifically disaggregates tau fibrils, or if it affects amyloid fibrils more broadly. We observed that CNS-11 and its chemical analog CNS-11g have a robust effect on alpha-synuclein fibrils. In this current study we characterize these effects using in vitro, in cellulo, in vivo, and in silico approaches.

### CNS-11 and CNS-11g Disaggregate Recombinant Alpha-Synuclein Fibrils In Vitro

We first aimed to characterize the ability of CNS-11 and CNS-11g to disaggregate alpha-synuclein fibrils in vitro. Both compounds were incubated with equimolar ratios of recombinant alpha-synuclein fibrils (100 μM:100 μM) for 48 hours at 37° C. in 1×PBS. After incubation, Thioflavin T (ThT), a fluorescent marker of amyloid fibrils, was added to the samples and ThT fluorescence was measured. EGCG was used as a positive control for comparison. A reduction in ThT signal is observed for all three compounds tested (FIG. 5a), indicating a reduction in fibril amount. Next, transmission electron microscopy (EM) was performed for each sample. Representative EM images shown in FIG. 5b demonstrate a qualitative reduction in fibril count for samples treated with CNS-11 and CNS-11g. To obtain a quantitative evaluation of fibril reduction, fibrils were first incubated for 72 hours with compound, then 10 micrographs from random grid points were taken for each sample, and the numbers of fibrils per image were counted (FIG. 5c). We observe a reduction in number of imaged fibrils for both compounds, particularly for CNS-11g. Western blot analysis, staining for alpha-synuclein, was performed for fibril samples incubated with compounds for 48 hours (FIG. 5d). For untreated alpha-synuclein fibrils, we see a prominent band at 14 kDa corresponding with protein monomer. We also observe a large smear of higher molecular weight alpha-synuclein species, likely corresponding to oligomers or small fibrils, and a dark band at the top of the gel likely from larger fibrils. When treated with compounds, particularly CNS-11g, we observe a reduction in the higher molecular weight species, and conversion to bands that primarily correspond to the smaller monomer, dimer and trimer species. Chemical structures of both CNS-11 (N-mesityl-2-(3-oxoindeno[1,2,3-de]phthalazin-2(3H)-yl)acetamide) and CNS-11g (2-(4-Benzyl-1-oxo-2(1H)-phthalazinyl)-N-(2,6-dimethylphenyl)acetamide) are shown in FIG. 5e, highlighting their similar chemical structures, low number of hydrogen bond acceptors and donors, and low number of rotatable bonds (Supp. Table 1 in Murray et al.). These physical and chemical properties are more predictive of oral bioavailability and central nervous system penetration compared to EGCG, making them more viable candidates for further drug development.

### CNS-11 and CNS-11g Prevent Intracellular Seeding of Alpha-Synuclein Fibrils and Mitigate Alpha-Synuclein Cytotoxicity

Having established the ability of CNS-11 and CNS-11g to disaggregate alpha-synuclein fibrils in vitro, we next sought to test both compounds in cellular models. It is thought that alpha-synuclein pathology propagates throughout the brain via a prion-like seeding mechanism²⁰. Alpha-synuclein aggregates in one cell can fragment and spread to neighboring cells, where they seed the aggregation of soluble protein. Molecules able to disaggregate pre-formed fibrils may be able to interrupt this process. To that end, we assessed if both compounds could mitigate intracellular seeding of αSyn fibrils using alpha-synuclein biosensor cells-HEK293T cells expressing A53T mutant alpha-synuclein fused with yellow fluorescent protein (YFP). At baseline, the fluorescently labeled alpha-synuclein remains soluble within the cell, visible as diffuse fluorescence. However, the liposome-mediated transduction of exogenous αSyn fibrils into the cells induces aggregation of the endogenous protein, and the aggregates can be visualized and quantified as fluorescent puncta within the cell.

CNS-11 and CNS-11g at various concentrations were incubated with recombinant alpha-synuclein fibrils for 48 hours, then transduced into the biosensor cells. We imaged the cells 48 hours after transduction of the samples and quantified the number of fluorescent puncta per sample well. For samples treated with fibrils alone (FIG. 6a), numerous bright puncta can be seen throughout the cells. Conversely, without any fibrils added, no puncta can be seen. Incubation of compounds with fibrils at a final compound concentration of 100 nM-10 M resulted in a dose-dependent reduction in aggregates. We used EGCG as a positive control, which also showed a dose-dependent reduction (FIG. 6b-d). For CNS-11, we observed an initial increase of seeding at low compound concentrations, then a sharp reduction in seeding at higher compound concentrations. The mechanism of this is unclear but may be a result of incomplete disaggregation/fragmentation of the fibrils at the low concentration producing a greater amount of seeding, whereas more complete disaggregation is occurring at the higher concentrations.

Alpha-synuclein aggregates, particularly oligomers, are known to be cytotoxic to neurons⁴. Thus, we next determined if disaggregation of alpha-synuclein aggregates by our compounds mitigates their cytotoxicity. Recombinant alpha-synuclein fibrils were again incubated with various concentrations of CNS-11 and CNS-11g for 48 hours. The fibril and compound mixtures were then added to cultured Neuro-2a (N2a) neuronal cells, to a final alpha-synuclein fibril concentration of 1 μM. A 3-(4,5-dimethylthiazol-2-yl)-2,5-diphenyltetrazolium bromide (MTT) dye reduction cell viability assay of the treated N2a cells reveals CNS-11g recovers the cytotoxicity of the alpha-synuclein fibrils at sub-stoichiometric concentrations. However, CNS-11 showed no rescue of toxicity at any concentration (FIG. 6e-f).

Compounds Disaggregate MSA Brain-Derived Alpha-Synuclein Fibrils and Prevent their Seeding

Having found that CNS-11 and CNS-11g disaggregate recombinant alpha-synuclein fibrils, we then investigated the efficacy of each compound on brain-derived alpha-synuclein fibrils. Post-mortem alpha-synuclein fibrils were extracted and purified from the brain of a patient with MSA. The presence of fibrils in the extract was confirmed by electron microscopy (FIG. 3a). CNS-11 and CNS-11g were incubated with the brain-derived fibrils for up to 3 days. Disaggregation of fibrils was observed for each compound. As shown in FIG. 7b, for CNS-11g treated MSA fibrils, disaggregation of a fibril was directly observed by electron microscopy (see red and white arrows). As with the recombinant fibrils, we next aimed to quantitatively assess brain-derived fibril disaggregation by EM. Compounds were incubated with MSA fibrils for 3 days, and fibrils were quantified by EM daily, with 10-15 images taken per experimental replicate. We observed a reduction in average number of imaged fibrils for both CNS-11 and CNS-11g compared to control (FIG. 7c).

Next, we tested the efficacy of the compounds to mitigate the intracellular seeding of MSA fibrils in biosensor cells. Brain-derived MSA fibrils robustly seed intracellular alpha-synuclein in HEK293T biosensor cells²¹. CNS-11, CNS-11g, and EGCG at various concentrations were incubated with MSA brain fibrils for 48 hours, sonicated, then transduced onto cells. Cells were imaged 48 hours and the number of fluorescent puncta per sample well was quantified. A very significant reduction in intracellular seeding is observed for all three compounds at sub micromolar concentrations, with both CNS-11 and CNS-11g showing efficacy comparable to the EGCG control. Together, these data demonstrate that these compounds can disaggregate MSA brain-derived fibrils in vitro, and fibrils pre-treated with CNS-11 and CNS-11g are no longer competent at seeding the intracellular aggregation of alpha-synuclein.

CNS-11 and CNS-11g Prevent Aggregation of Alpha-Synuclein in C. elegans

Given our findings that both CNS-11 and CNS-11g can reduce alpha-synuclein aggregates in vitro and in cellular models, we next studied their efficacy using an in vivo model of alpha-synuclein pathology. The DDP1 C. elegans strain overexpresses alpha-synuclein fused with yellow fluorescent protein (YFP) and cyan fluorescent protein (CFP)^{22,23}. FRET-positive fluorescent alpha-synuclein aggregates can then be visualized and quantified in the adult worms. DDP1 worms were synchronized (see Methods) then the L1 larvae were grown on plates treated with CNS-11 and CNS-11g at 100 μM. PBS was used as a vehicle control. At day 6 of adulthood, the worms were visualized, and the number of aggregates were quantified by fluorescent microscopy. As shown in FIG. 4a, numerous fluorescent aggregates are visible in the head region of vehicle treated (“no inhibitor”) worms. Treatment with either compound resulted in a reduction of total aggregates, as quantified in FIG. 8b. Treated worms were also homogenized for protein analysis by Western blot. Homogenate for each experimental condition was separated into insoluble pellet fractions (P) and soluble supernatant (S) fractions. For untreated worm homogenate, the majority of alpha-synuclein is found in the insoluble pellet. Treatment with CNS-11 and CNS-11g results in solubilization of the alpha-synuclein, presumably from disrupting the formed aggregates (FIG. 8c).

### Detection and Quantification of CNS-11 and CNS-11G in Serum and Brain Tissue in Mice

Based on their biophysical properties both CNS-11 and CNS-11g are predicted to have favorable brain permeability. Nevertheless, it was necessary to experimentally demonstrate that this prediction was correct. The compounds were administered by tail vein injection at a dose of 0.5 mg/kg of body weight to C57BL/6J mice (n=3 for each compound). Brain and plasma samples were collected 1 hour after dosing. To detect and quantify the drugs we used a sensitive, selective, and specific liquid chromatographic/tandem mass spectrometric multiple reaction monitoring (LC-MS/MRM) assay. The sample extraction protocol for plasma and brain was optimized with spiking experiments in which the authentic compounds were added to plasma and brain from drug-naive mice. One hour following administration both CNS-11 and CNS-11g were measured in the plasma of treated wild-type mice with a range of concentrations of 5.0-28.4 nM for CNS-11 and 10.4-58.8 nM for CNS-11g (FIG. 9a). In addition, both CNS-11 and CNS-11g demonstrated brain permeability. Compounds were measured in brain tissue with a range of concentrations of 6.9-21.5 ng/g of brain for CNS-11 and 4.2-26.1 ng/g of brain for CNS-11g (FIG. 9b). These data confirm the prediction that both CNS-11 and CNS-11 g are brain penetrating in vivo.

### Molecular Dynamics Simulations of CNS-11 and CNS-11g Reveal Possible Mechanism of Alpha-Synuclein Fibril Disaggregation

Lastly, we sought to determine a putative binding site for CNS-11 and CNS-11 g to alpha-synuclein fibrils, and to elucidate the compounds' possible mechanism for fibril disaggregation. To accomplish this, we performed multiple molecular dynamics (MD) simulations of each compound bound to four possible binding sites along the fibrils surface, as well as for an unbound control fibril (FIG. 9a), using the alpha-synuclein recombinant fibril structure (PDB: 6cu7)⁷. MD simulations were carried out for a total of 40 ns. For each simulation, we measured the distance between the top layer of the alpha-synuclein fibril and the layer just beneath it. In most amyloid fibril structures a distance of ˜4.8 Å is seen between fibril layers, corresponding to beta-strand separation within the beta-sheet. Thus, if fibril disaggregation occurs, we expect the inter-layer spacing to increase beyond 4.8 A. For each of the four binding sites assessed, we analyzed the inter-layer distance between Ca atoms of the residues adjacent to the compound binding site, for both CNS-11 and CNS-11g (FIG. 9b). For both Site 1 and Site 2, found at the N-terminus of the fibril core, we observe relative stability of the unbound fibril structure at residues 41-45, with little fluctuation between inter-layer distance. However, with addition of CNS-11 at Site 1, the inter-layer distances quickly increase (FIG. 9c). Similarly, both CNS-11 and CNS-1 g greatly disrupt intra-strand spacing when bound to Site 2. In contrast, we do not observe large inter-strand distance increases in any of the residues adjacent to Site 3 or Site 4 (FIG. 9c) when bound to CNS-11 or CNS-11g. From these findings we conclude that a possible mechanism of fibril disaggregation by CNS-11 and CNS-11g is through destabilization of the N-terminal residues (Gly41-Lys45) of the fibril core, either bound to Site 1 or Site 2, and less likely through the C-terminus (Sites 3 and 4). While this provides an initial putative mechanism by which these compounds are functioning, further structural and biophysical characterization will be needed to fully understand their effects on fibril architecture.

### Methods

### Expression and Purification of Alpha-Synuclein

The alpha-synuclein construct was transformed into BL21(DE3) Gold E. Coil. Protein was expressed following inoculation of a 30 mL starter with from colonies after selection with ampicillin. 6 L LB media was inoculated with the starter culture and grown to an OD600 of 0.6-0.8 shaking at 220 rpm at 37° C. Protein expression was induced by the addition of 500 μM IPTG, then cells were grown for an additional 3 hours. Cells were centrifuged at 4000 rpm for five minutes, and cell pellet was resuspended in 100 mL of lysis buffer (100 mM Tris-HCl pH 8.0, 1 mM EDTA) and pellet was sonicated on ice to lyse cells. Cell lysate was centrifuged for 30 minutes at 15,000 rpm. 0.22 g/mL ammonium sulfate was added to lysate for 30 minutes, then centrifuged for 30 minutes at 15,000 rpm. The pellet was resuspended in 80 mL of 20 mM Tris pH 8.0. Solution was dialyzed in 20 mM Tris pH 8.0 overnight. A HiPrep Q HP column (GE Healthcare) was then used to purify the protein. A gradient 0-100% of buffer A (20 mM Tris pH 8.0) to buffer B (20 mM Tris pH 8.0, 500 mM NaCl) was used over a volume of 100 mL. Fractions were collected and then ran on size exclusion G3000 column (Tosoh Bioscience), using a buffer of 100 mM sodium sulfate, 25 mM sodium phosphate, and 1 mM sodium azide at pH 6.5. The protein was then dialyzed in 100 mM sodium sulfate and 25 mM sodium phosphate overnight, with two exchanges of buffer. Protein was the concentrated and flash frozen for storage using liquid nitrogen. Concentration of protein was determined by Pierce BCA assay (Thermo #23225).

### Generation of Recombinant Alpha-Synuclein Fibrils

50 mM of recombinant alpha-synuclein in 1×PBS was added to several wells of a Nunc black optical bottom plate (Thermo Scientific) to a total volume of 100 uL per well with a single PTFE bead (0.125-inch diameter) per well. The plate was then agitated using a Torrey Pines floor shaker on maximum speed for 96 hours at 37° C. The presence of fibrils within the samples was confirmed using transmission electron microscopy (see below) prior to use in experimental assays.

### Transmission Electron Microscopy

For all transmission electron microscopy (TEM), 6 μL of sample were added onto 400 mesh Formvar Carbon film copper mesh grids (Electron Microscopy Sciences), then incubated for four minutes. 6 μL of 2% uranyl acetate solution was then used to stain the grids. After 2 minutes, excess solution was blotted off and grids were left to dry for a minimum of 30 minutes. TEM images were taken on a Tecnai 12 transmission electron microscope. For quantitation of fibrils, n=20 TEM images were taken per experimental condition from random points throughout the grid. Fibril counts were manually quantified per image in a blinded fashion.

### Disaggregation Assays

For all disaggregation assays, 50 mM stocks of CNS-11 and CNS-11g in DMSO were used to make working concentrations of compound in 1×PBS. Compounds were incubated quiescently with fibril samples (both recombinant and brain-derived) for 72 hours at 37° C.

### Western Blot Analysis

Samples were loaded onto a NuPAGE 12% Bis-Tris pre-cast protein gel and ran for 35 minutes at 200 V. To transfer protein from the gel to a nitrocellulose membrane an iBLOT2 dry blotting system was used. The membrane was blocked in TBST with 5% milk for 1 hour, then washed three times with TBST. The membrane was incubated with the primary antibody (anti-alpha synuclein MJFR1 (Abcam, cat #ab138501)) 1:5000 dilution in 5% milk/TBST solution) for 1 hour, washed three times with TBST, incubated with the horseradish peroxidase-conjugated secondary antibody (goat anti-rabbit IgG H+L (Invitrogen; cat #A27036, lot #2116291); 1:4000 Dilution in 5% milk/TBST), and washed three times in TBST. Signal was detected with Pierce ECL Plus Western Blotting Substrate (Cat #32132), and blot was imaged with a Pharos FX Plus Molecular Imager.

For C. elegans experiments, the same protocol was used, except following the treatment period worms were harvested, flash frozen, then homogenized for protein extraction. The membrane was washed with TBST overnight before repeating the Western blot analysis for beta-actin (primary antibody anti-beta-actin (C4) (Santa Cruz Biotechnology, cat #sc-47778, lot #J1119; 1:500 dilution in 5% milk/TBST); horseradish peroxidase-conjugated secondary antibody (goat polyclonal anti-mouse IgG (Abcam; cat #ab205719, lot #GR3271082-2); 1:5000 dilution in 5% milk/TBST).

### 3-(4,5-dimethylthiazol-2-yl)-2,5-diphenyltetrazolium bromide (MTT) dye reduction cell viability assay

Neuro-2a cells (ATCC cat. #CCL-131) were cultured in MEM media (Life Technologies cat. #11095-080) with 10% FBS (Life Technologies cat. #10437010) and 1% pen-strep (Life Technologies cat. #15140122) in 5% C02 incubator at 37° C. N2a cells were plated onto clear 96-well plates (Costar cat. #3596) at 5000 cells/well in 90 uL culture media for 24 hours. Recombinant alpha-synuclein fibril samples were co-incubated with and without CNS-11 or CNS-11g in 10 uL volume overnight at 37° C., then added to the N2a cells (final fibril concentration of 1 μM). All experiments were performed in triplicate. After incubation for 24 hours, 20 μL of Thiazolyl Blue Tetrazolium Bromide MTT dye (Sigma; 5 mg/mL stock in DPBS) was added to each well then incubated for 3.5 hours at 37° C. Removal from the incubator and replacement of well media with 100 μL of 100% DMSO halted the assay. Absorbance was measured at 570 nm using a SpectraMax M5 reader. A background reading at 700 nm was subtracted from the 570 nm reading. Well readings were normalized to vehicle alone treated cells (designated as 100% viable) and cells treated with 100% DMSO (designated as 0% viable).

### Biosensor Cell Seeding Assays

HEK293T biosensor cells stably expressing YFP-fused A53T mutant alpha-synuclein, developed and provided by the lab of Marc Diamond at UTSW were used. Cells were grown in DMEM (Life Technologies, cat. 11965092) with FBS (10% vol/vol; Life Technologies, cat. A3160401), penicillin/streptomycin (1%; Life Technologies, cat. 15140122), and Glutamax (1%; Life Technologies, cat. 35050061), at 37° C. and 5% CO2 in a humidified incubator. Compounds were incubated with recombinant or patient-derived alpha-synuclein fibrils for 48 hours in OptiMEM media then applied to ˜70% confluent biosensor cells. Prior to adding to cells, the coincubated compound/fibril solution was sonicated for 5 minutes in a cuphorn water bath, then mixed for 20 minutes with Lipofectamine 2000 in OptiMEM (1:20 dilution) for 20 minutes. 10 uL of the inhibitor/fibril+Lipofectamine mixture was added to 90 uL of cells plated in black 96-well tissue culture plates in triplicate for each concentration of compound tested. The number of seeded aggregates was quantified using a Celigo Image Cytometer (Nexcelom) in the YFP channel. Images were processed in ImageJ, background fluorescence from unseeded cells was subtracted, and the number of particles per image were counted using the Particle Analyzer function. The quantity of aggregates in each well was normalized to cell confluence. Standard deviation between triplicates and a nonlinear regression curve was used to calculate IC₅₀ values for dose response curves. High quality fluorescent images were obtained using a ZEISS Axio Observer D1 fluorescence microscope in the YFP channel.

Extraction of Alpha-Synuclein Fibrils from Patient Brain Tissue

Extraction of sarkosyl-insoluble alpha-synuclein fibrils from neuropathologically confirmed brain samples of patients diagnosed with MSA was performed using the method previously described by Schweighauser et al. without any modifications¹³. The presence of fibrils in each extract was confirmed by TEM prior to use in experiments. The presence of alpha-synuclein was confirmed by immunoblotting using anti-alpha synuclein MJFR1 antibody (Abcam, cat #ab138501).

C. elegans Experiments

The DDP1 (uonExl [unc-54::αSyn::CFP+unc-54::αSyn::YFP(Venus)]) strain was acquired from the Caenorhabditis Genetics Center (CGC) and used for experiments. C. elegans were grown and maintained using standard conditions. Worms were synchronized using hypochlorite bleaching, hatched ovemight in M9 media (5 g/l NaCl, 6 g/l Na2HPO₄, 3 g/l KH₂PO₄, 1 μM MgSO₄) at 17° C., then cultured on plates with nematode growth medium (NGI; 17 g/l agar, 2.5 g/l peptone, 3 g/l NaCl 1 mM CaCl₂, 1 mM MgSO₄, 25 mM KH₂PO₄ pH 6, 5 μg/ml cholesterol) seeded with OP50 E. coli. Strains were maintained at 17° C. For inhibitor treatment conditions, CNS-11 and CNS-11g were diluted in 1×PBS to final compound concentration of 100 μM and the solution was added to plates. Treatment plates were then seeded with heat treated OP50 (30 min at 65° C.), according to the “NGM dead” method as previously published³⁶. Synchronized L1 worms were then added to the treatment plates and grown for 7 days. PBS was used as a control condition. 75 μM FUDR was added on the third day of growth. For imaging, worms were mounted onto 5% agar pads on glass slides, immobilized with 1% NaN3 solution, and imaged by fluorescent microscopy (GFP channel) using a ZEISS Axio Observer D1 fluorescence microscope. Alpha-synuclein aggregates in the head region were quantified.

### Animal Experiments.

All animal experiments were approved by the UCLA Animal Research Committee and performed under oversight of the Division of Laboratory Animal Medicine (DLAM). C57BL/6J mice (Jackson Laboratories: JAX:000664) were housed on a 12-hr light/dark schedule.

Sample preparation for MRM analysis.

Mice were injected intravenously with CNS-11 (n=3) or CNS-11g (n=3) at a concentration of 0.5 mg/kg, and sacrificed by perfusion 1 hour post-injection. Blood was collected by cardiac puncture and plasma was recovered as the supernatant after centrifugation (3000×g, 10 m) and stored frozen. Brains were collected by standard dissection and immediately frozen.

To measure the average drug concentrations across the entire brains each left hemisphere (average wet weight 250 mg) was processed. The samples were spiked with the internal standard (IS, CNS-11F, 15 pmol in 15 μL of acetonitrile) and homogenized with a probe sonicator (Kontes microsonic cell disrupter, 30 sec) after the addition of acetonitrile/water (4:1, v/v, 2500 μL). The homogenates were divided evenly into three polypropylene microcentrifuge tubes (met) for triplicate measurements of each sample. Following centrifugation (16,100g, 5 min), the supernatants were transferred to clean mets and dried in a vacuum centrifuge. The pellets were treated with acetonitrile (25 μL) with vigorous mixing, followed by water (25 μL) with more vigorous mixing, followed by more water (50 μL) for a total volume of 100 μL. The samples were mixed again, centrifuged (16,100g, 5 min) and the supernatants were transferred to HPLC injector vials. With each batch of tissue samples, a series of standards were prepared in which CNS-11 and CNS-11g (0, 1, 2.5, 5, and 10 μmol, each in duplicate) were added to drug-naive perfused murine brain tissue (100 mg per tube), along with the same amount of IS (5 pmoles). These samples were processed as described above.

Plasma samples (50 μL, in duplicate for each mouse) were spiked with the internal standard (IS, CNS-11F, 5 pmol in 5 μL of acetonitrile), and vigorously mixed after the addition of acetonitrile (500 μL). Following centrifugation (16,100g, 5 min), the supernatants were transferred to clean mcts and dried in a vacuum centrifuge. The pellets were treated with acetonitrile (25 μL) with vigorous mixing, followed by water (25 μL) with more vigorous mixing, followed by more water (50 μL) for a total volume of 100 ul. The samples were mixed again, centrifuged (16,100g, 5 min) and the supernatants were transferred to HPLC injector vials. With each batch of plasma samples, a series of standards were prepared in which CNS-11 and CNS-11g (0, 1, 2.5, 5, and 10 μmol, each in duplicate) were added to drug-naive murine plasma (50 μL per tube), along with the same amount of IS (5 pmoles). These samples were processed as described above.

Combined Liquid Chromatography-Tandem Mass Spectrometry with Multiple Reaction Monitoring (MRM).

Aliquots of each sample (2 μL) were injected onto a reverse phase HPLC column (Phenomenex Kinetex C18, 2.6 μm, 100×2.1 mm) equilibrated in solvent A 5 (water/formic acid, 100/0.1, v/v) and eluted (100 μL/min) with a linearly increasing concentration of solvent B (acetonitrile/formic acid, 100/0.1, v/v; min/% B: 0/5, 5/5, 30/100, 32/5, 40/5). The effluent from the column was directed to an electrospray ion source (Agilent Jet Stream) connected to a triple quadrupole mass spectrometer (Agilent 6460) operating in the positive ion tandem mass spectrometric multiple reaction monitoring (MRM) mode in which the intensity of the transition of preselected parent ions to preselected fragment ions was recorded after signal optimization (collision energy, fragmentor voltage and collision cell accelerator voltage) with instrument manufacturer-supplied software (Mass Hunter). Two transitions were monitored for each drug: m/z 398.1→249.2 (for quantitation) and 277.2 (for confirmation) for CNS-11G (retention time 23.3 min); m/z 396.2-233.1 (for quantitation) and 261.0 (for confirmation) for CNS-11 (retention time 24.3 min), and one transition was monitored for the IS; m/z 412.3-249.2 (retention time 24.3 min). Peak areas were integrated and recorded, and a curve was constructed from the data obtained from the standards in which the ratio of drug peak area/IS peak area was plotted against the amount of drug in each sample. The amount of each drug in the samples was then derived by interpolation from the curve.

### Computational Models and Molecular Dynamics

Three dimensional structures of CNS-11 and CNS-11g were generated using OpenBabel. Both compounds were docked to four unique locations along the alpha-synuclein fibril structure (PDB code: 6cu7) using AutoDock vina. Top scoring bound poses were used as starting trajectories for subsequent molecular dynamics (MD) experiments. MD was performed using GROMACS version 2020 using a CHARMM36 force field. Ligand topologies for CNS-11 and CNS-11g were generated using the CGenFF server. Hydrogen atoms were added to all structures using Avogadro. The fibril alone and fibril/compound complex structures were solvated using a 1 nm dodecahedron water box. Chloride ions were added to generate a charge neutral system. The system was first energy minimized, then temperature and pressure equilibrated under and NVT ensemble for 100 ps then NPT for an additional 100 ps. Production MD runs were performed for 40 ns for each system. Analysis of molecular distances was performed in GROMACS and manually visualized with PyMol. Images of molecular models were generated with UCSF Chimera.

### DISCUSSION

Current treatment options for synucleinopathies, including PD, MSA, and DLB, are capable only of symptom management; there are no available therapies for any synucleinopathy that modify disease progression. PD is the second most common neurodegenerative disease, and with the aging of our population, the need for therapeutic options is becoming increasingly urgent. Alpha-synuclein was determined to be the primary component of Lewy bodies over twenty years ago²⁴. Since that discovery, the aggregation of alpha-synuclein has been identified as key component in the pathology of these diseases²⁵. Considerable effort has been invested in the screening and identification of compounds able to either inhibit the formation of or disassemble alpha-synuclein aggregates¹⁵. High-throughput small molecule screens have identified several promising compounds, including SynuClean-D²⁶, anle138b²⁷, BIOD303²⁸. Recently the carotenoid crocin was demonstrated to both inhibit aggregation and disassemble mature alpha-synuclein fibrils²⁹.

As previously mentioned, CNS-11 was originally identified from a screen of compounds capable of disaggregating tau paired helical filaments (PHFs), the primary tau aggregate in Alzheimer's disease. This screen was guided by the structure of EGCG bound to disaggregating tau PHFs. EGCG is a known potent disaggregator of amyloid fibrils, including tau¹⁸ and alpha-synuclein³⁰. Unfortunately, its highly polar chemical composition greatly reduces its bioavailability, diminishing its promise as a potential therapeutic³¹. Our aim was to identify compounds capable of disaggregating fibrils like EGCG, but with more drug-like biophysical properties. CNS-11 and CNS-11g were identified from a library of compounds with increased probability of blood-brain barrier penetration, a key hurdle in the development of therapeutics for neurodegenerative disease. Both CNS-11 and CNS-1 g lack the numerous hydroxyl groups of EGCG, and have low polar surface areas and molecular weights predictive of central nervous system penetration³², making them more promising therapeutic leads than EGCG. Both CNS-11 and CNS-11g also satisfy the Lipinski and Verber rules of drug-like compounds, which EGCG violates (Supp. Table 1 in Murray et al.) and are demonstrated to penetrate brain tissue in mice following tail-vein injection (FIG. 9).

Here, we have demonstrated that CNS-11 and CNS-11g can disaggregate alpha-synuclein and that treatment of fibrils with either compound also reduces the seeded aggregation of alpha-synuclein. This reduction has important implications, as the spread of alpha-synuclein pathology in the brain is thought to occur through a templated seeding mechanism. A possible pitfall of disaggregation as a therapeutic mechanism is that it may fragment fibrils, thereby producing more seeds that can propagate and promote disease pathology instead of mitigating it. For example, Nachman et al. recently demonstrated that disassembly of tau fibrils by the chaperone protein Hsp70 can generate seeding-competent species³³. Thus, for the investigation of any new potential therapeutic that modifies alpha-synuclein aggregates, it is essential to assess its effects on seeding. In the case of CNS-11, we do observe an increase in seeding of recombinant alpha-synuclein fibrils (FIG. 6) at low compound concentrations, which may represent incomplete disaggregation of the fibrils. This is not seen for CNS-11g, which may indicate that CNS-11g converts the fibrils into a monomeric form or small multimeric form incapable of seeding. We do not observe any enhancement of seeding from MSA brain-extracted fibrils for either compound, and instead see a dose-dependent reduction in seeding even at low concentrations. Nonetheless, effects on seeding at a range of doses should be an important consideration during advancement of molecules that target alpha-synuclein, including the compounds presented in this work.

The cell has existing machinery to combat protein aggregation and disassemble pathologic fibrils once they form. Chaperone proteins Hsc70 with DNAJB1 and Apg2 are able revert fibrillar alpha-synuclein back to soluble monomer, as well as Hsp70 with DNAJB1³⁴. Recent mechanistic studies have revealed this disassembly of fibrils may occur through the removal of monomer units directly from the fibril ends³⁵. Our molecular dynamics experiments of CNS-11 and CNS-11g bound to the alpha-synuclein fibril core may reveal a similar mechanism of disassembly (FIG. 8). First, our simulations of the compounds docked to four potential binding sites reveal areas near the N-terminus of the fibril to be the most destabilized by the compounds. For the simulations that showed fibril disaggregation, we observe that the top layers of the fibrils are the most destabilized even though the simulations are initiated with the compounds bound centrally along the side of the fibril. Thus, CNS-11 and CNS-11g may be acting in a similar way to the chaperone assemblies, peeling off the unstable end layers of the fibrils (Supp. FIG. 1 in Murray et al.). Based on our calculations, the mechanism of binding for either compound to the fibril appears to be driven primarily by interactions with hydrophobic sidechains, as well as hydrogen bonding with backbone amide groups. However, further structural and biochemical studies will be needed to fully understand the mechanism of compound action.

Here we have demonstrated the ability of two compounds to disassemble alpha-synuclein with both in vitro and in vivo models. We also show the compounds are effective on alpha-synuclein fibrils directly extracted from patient brain tissue and are capable of penetrating living brain tissue in mice. Further validation will be needed to establish their therapeutic efficacy, but these preliminary results demonstrate potential of these compounds as leads for future drug development towards the treatment of svnucleinopathies. Given that the compounds also have effects on tau aggregates and the biophysical properties of a drug-like compound, CNS-11 and its related analogs may also have future promise in treatment of other diseases involving aberrant protein aggregation.

### Example 2 References

1. Barker, R. A. & Williams-Gray, C. H. Review: The spectrum of clinical features seen with alpha synuclein pathology. Neuropathol. Appl. Neurobiol. 42, 6-19 (2016).
2. Goedert, M. Alpha-synuclein and neurodegenerative diseases. Nat. Rev. Neurosci. 2, 492-501 (2001).
3. Meade, R. M., Fairlie, D. P. & Mason, J. M. Alpha-synuclein structure and Parkinson's disease—lessons and emerging principles. Mol. Neurodegener. 14, 29 (2019).
4. Winner, B. et al. In vivo demonstration that α-synuclein oligomers are toxic. Proc. Natl. Acad. Sci. 108, 4194-4199 (2011).
5. Fellner, L., Jellinger, K. A., Wenning, G. K. & Stefanova, N. Glial dysfunction in the pathogenesis of α-synucleinopathies: emerging concepts. Acta Neuropathol. (Berl.) 121, 675-693 (2011).
6. Bendor, J., Logan, T. & Edwards, R. H. The Function of α-Synuclein. Neuron 79, (2013).
7. Li, B. et al. Cryo-EM of full-length α-synuclein reveals fibril polymorphs with a common structural kernel. Nat. Commun. 9, 1-10 (2018).
8. Guerrero-Ferreira, R. et al. Cryo-EM structure of alpha-synuclein fibrils. eLife 7, e36402 (2018).
9. Li, Y. et al. Amyloid fibril structure of α-synuclein determined by cryo-electron microscopy. Cell Res. 28, 897-903 (2018).
10. Boyer, D. R. et al. Structures of fibrils formed by α-synuclein hereditary disease mutant H50Q reveal new polymorphs. Nat. Struct. Mol. Biol. 26, 1044-1052 (2019).
11. Boyer, D. R. et al. The α-synuclein hereditary mutation E46K unlocks a more stable, pathogenic fibril structure. Proc. Natl. Acad. Sci. U.S.A 117, 3592-3602 (2020).
12. Tuttle, M. D. et al. Solid-State NMR Structure of a Pathogenic Fibril of Full-Length Human α-Synuclein. Nat. Struct. Mol. Biol. 23, 409-415 (2016).
13. Schweighauser, M. et al. Structures of α-synuclein filaments from multiple system atrophy. Nature 585, 464-469 (2020).
14. Luk, K. C. et al. Pathological α-Synuclein Transmission Initiates Parkinson-like Neurodegeneration in Nontransgenic Mice. Science 338, 949-953 (2012).
15. Fields, C. R., Bengoa-Vergniory, N. & Wade-Martins, R. Targeting Alpha-Synuclein as a Therapy for Parkinson's Disease. Front. Mol. Neurosci. 12, (2019).
16. Sangwan, S. et al. Inhibition of synucleinopathic seeding by rationally designed inhibitors. eLife 9, e46775 (2020).
17. Bieschke, J. et al. EGCG remodels mature alpha-synuclein and amyloid-beta fibrils and reduces cellular toxicity. Proc. Natl. Acad. Sci. U.S.A 107, 7710-7715 (2010).
18. Sonawane, S. K. et al. EGCG impedes human Tau aggregation and interacts with Tau. Sci. Rep. 10, 12579 (2020).
19. Seidler, P. et al. CryoEM reveals how the small molecule EGCG binds to Alzheimer's brain-derived tau fibrils and initiates fibril disaggregation. bioRxiv 2020.05.29.124537 (2020) doi:10.1101/2020.05.29.124537.
20. Masuda-Suzukake, M. et al. Prion-like spreading of pathological α-synuclein in brain. Brain 136, 1128-1138 (2013).
21. Prusiner, S. B. et al. Evidence for α-synuclein prions causing multiple system atrophy in humans with parkinsonism. Proc. Natl. Acad. Sci. 112, E5308-E5317 (2015).
22. Bodhicharla, R. et al. Effects of α-synuclein overexpression in transgenic Caenorhabditis elegans strains. CNS Neurol. Disord. Drug Targets 11, 965-975 (2012).
23. Nagarajan, A. et al. A Fluorescence Resonance Energy Transfer Assay For Monitoring α-Synclein Aggregation in a Caenorhabditis Elegans Model For Parkinson's Disease. CNS Neurol. Disord. Drug Targets 14, 1054-1068 (2015).
24. Spillantini, M. G. et al. Alpha-synuclein in Lewy bodies. Nature 388, 839-840 (1997).
25. Lashuel, H. A., Overk, C. R., Oueslati, A. & Masliah, E. The many faces of α-synuclein: from structure and toxicity to therapeutic target. Nat. Rev. Neurosci. 14, 38-48 (2013).
26. Pujols, J. et al. Small molecule inhibits α-synuclein aggregation, disrupts amyloid fibrils, and prevents degeneration of dopaminergic neurons. Proc. Natl. Acad. Sci. 115, 10481-10486 (2018).
27. Wagner, J. et al. Anle138b: a novel oligomer modulator for disease-modifying therapy of neurodegenerative diseases such as prion and Parkinson's disease. Acta Neuropathol. (Berl.) 125, 795-813 (2013).
28. Moree, B. et al. Small Molecules Detected by Second-Harmonic Generation Modulate the Conformation of Monomeric α-Synuclein and Reduce Its Aggregation in Cells*. J. Biol. Chem. 290, 27582-27593 (2015).
29. Saffari, B. & Amininasab, M. Crocin Inhibits the Fibrillation of Human α-synuclein and Disassembles Mature Fibrils: Experimental Findings and Mechanistic Insights from Molecular Dynamics Simulation. ACS Chem. Neurosci. 12, 4037-4057 (2021).
30. Bieschke, J. et al. EGCG remodels mature α-synuclein and amyloid-D fibrils and reduces cellular toxicity. Proc. Natl. Acad. Sci. 107, 7710-7715 (2010).
31. Kanwar, J. et al. Recent advances on tea polyphenols. Front. Biosci. Elite Ed. 4,111.
32. Wager, T. T., Hou, X., Verhoest, P. R. & Villalobos, A. Moving beyond Rules: The Development of a Central Nervous System Multiparameter Optimization (CNS MPO) Approach To Enable Alignment of Druglike Properties. ACS Chem. Neurosci. 1, 435-449 (2010).
33. Nachman, E. et al. Disassembly of Tau fibrils by the human Hsp70 disaggregation machinery generates small seeding-competent species. J. Biol. Chem. 295, 9676 (2020).
34. Wentink, A. S. et al. Molecular dissection of amyloid disaggregation by human HSP70. Nature 587, 483-488 (2020).
35. Schneider, M. M. et al. The Hsc70 disaggregation machinery removes monomer units directly from α-synuclein fibril ends. Nat. Commun. 12, 5999 (2021).
36. Zheng, S.-Q., Ding, A.-J., Li, G.-P., Wu, G.-S. & Luo, H.-R. Drug Absorption Efficiency in Caenorhbditis elegans Delivered by Different Methods. PLoS ONE 8, (2013).

All publications mentioned herein (e.g. the references numerically listed above) are incorporated herein by reference to disclose and describe aspects, methods and/or materials in connection with the cited publications.

TABLE A
SMALL MOLECULES DISCOVERED TO FUNCTION AS DISAGGREGATORS OF AMYLOID FIBRILS.

| Name | Structure | Chemical name | Function |
|---|---|---|---|
| CNS11G | 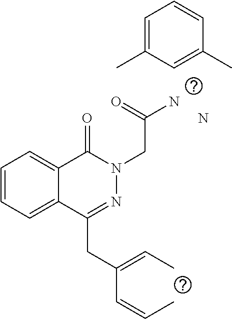 | 2-(4-benzyl-1-oxophthalazin-2-yl)- N-(2,6- dimethylphenyl)acetamide | Dissociate α- synuclein fibrils |
| MOL01 | 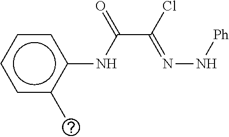 | 2-Chloro-N-(2,6- dichlorophenyl)benzamide | Dissociate α- synuclein fibrils |
| MOL06 | 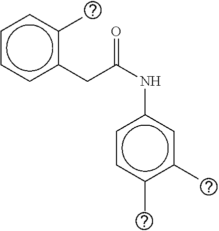 | N-(4-bromo-3-methylphenyl)-2- (2-chlorophenyl)acetamide | Dissociate α- synuclein fibrils |
| MOL18 | 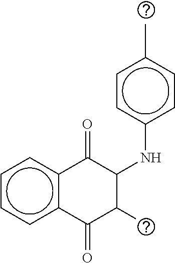 | 2-chloro-3-[(4- chlorophenyl)amino]naphthalene- 1,4-dione | Dissociate tau fibrils |
| CNS11 | 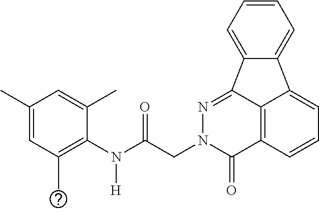 [SMILES: Cc1cc(C)c(NC(=O)Cn2nc3c4c(cccc4c2=O)-c2ccccc2-3)c(C)c1] | N-mesityl-2-(3- oxoindeno[1,2,3-de]phthalazin- 2(3H)-yl)acetamide | Dissociate tau fibrils |
| 1541 | 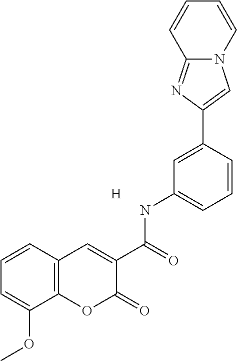 | N-(3-(imidazo[1,2-a]pyridin-2- yl)phenyl)-8-methoxy-2-oxo- 2H-chromene-3-carboxamide | Dissociate tau fibrils |

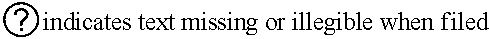

1. A composition of matter comprising at least one of:
CNS-2;
CNS-11;
CNS-11G;
CNS-12;
CNS-17;
MOL01;
MOL06;
MOL18; or
1541; and
a pharmaceutically acceptable carrier.

2. The composition of claim 1, further comprising amyloid fibrils.

3. The composition of claim 2, wherein the amyloid fibrils comprise tau amyloid fibrils or alpha-synuclein amyloid fibrils.

4. The composition of claim 3, wherein the CNS-2; CNS-11; CNS-11G; CNS-12; CNS-17; MOL01; MOL06; MOL18; or 1541 is bound to pharmacophores/sites on tau amyloid fibrils or alpha-synuclein amyloid fibrils that are bound by epigallocatechin gallate (EGCG).

5. The composition of claim 4, wherein the composition includes an agent:
selected to bind to tau fibrils along an amyloid fibril axis with a spacing of between 4 A and 5 A (e.g., ˜4.8 A); and/or
selected to form fibrils having a spacing along the fibril axis of between 4 A and 5 A (e.g., ˜4.8 A).

6. The composition of claim 3, wherein the tau amyloid fibrils or alpha-synuclein amyloid fibrils are disposed in an in vitro environment.

7. A method of making a pharmaceutical composition comprising combining at least one of:
CNS-2;
CNS-11;
CNS-11G;
CNS-12;
CNS-17;
MOL01;
MOL06;
MOL18; or
1541;
with a pharmaceutically acceptable excipient such that the pharmaceutical composition is made.

8. The method of claim 7, wherein the method includes disposing amyloid fibrils in the composition.

9. The method of claim 8, wherein the amyloid fibrils are selected to comprise tau amyloid fibrils or alpha-synuclein amyloid fibrils.

10. The method of claim 9, wherein the CNS-2; CNS-11; CNS-11G; CNS-12; CNS-17; MOL01; MOL06; MOL18; or 1541 is disposed in an aqueous solution and bound to pharmacophores/sites on tau amyloid fibrils or alpha-synuclein amyloid fibrils.

11. A method of identifying agents capable of binding amyloid fibrils comprising;
disposing fibrils in an aqueous solution in a container;
combining the fibrils with one or more test agents; and
observing the ability of the one or more test agents to bind the fibrils; wherein the method includes:
observing binding of the one or more agents to a pharmacophore on fibrils that is bound by epigallocatechin gallate (EGCG) such as site 1 binding clefts.

12. The method of claim 11, wherein the method includes at least one of:
a cryogenic electron microscopy method;
in silico screening of a library of drug-like small molecule compounds; and/or
observing the total binding energy of the test agent/fibril complex.

13. The method of claim 12, wherein the method includes cryogenically trapping a disaggregant (e.g., EGCG) to amyloid fibrils (e.g., tau fibrils extracted from patients' brains).

14. A method of inhibiting the aggregation of and/or facilitating disaggregating fibrils of tau or alpha-synuclein proteins comprising combining the fibrils of tau or alpha-synuclein proteins with at least one of CNS-2; CNS-11; CNS-11G; CNS-12; CNS-17; MOL01; MOL06; MOL18; or 1541 such that the aggregation of and/or facilitating the disaggregating fibrils of tau or alpha-synuclein proteins is inhibited.

15. A method for treating a subject having an amyloid disease characterized by aggregation of tau amyloid fibrils or aggregation of alpha-synuclein amyloid fibrils, comprising administering to the subject an effective amount of a pharmaceutical composition comprising at least one of:
CNS-2;
CNS-11;
CNS-11G;
CNS-12;
CNS-17;
MOL01;
MOL06;
MOL18; or
1541; and
a pharmaceutically acceptable excipient.

16. The method of claim 15, wherein the amyloid disease characterized by aggregation of Tau protein is Alzheimer's disease.

17. The method of claim 15, wherein the amyloid disease characterized by aggregation of alpha-synuclein protein is Parkinson's disease.

18. A method of inhibiting formation of tau fibrils comprising:
combining tau with a composition at least one of:
CNS-2; CNS-11; CNS-12; CNS-17; MOL18 and 1541; and
a pharmaceutically acceptable carrier; and
allowing the CNS-2; CNS-11; CNS-12; CNS-17; MOL18 and 1541 to bind to tau; so that Tau fibril formation is inhibited.

19. The method of claim 18, wherein the CNS-2; CNS-11; CNS-11G; CNS-12; or CNS-17 is combined with tau in vivo.

20. The method of claim 19, wherein inhibiting formation of tau fibrils in vivo inhibits development or progression of a tauopathy in an individual.
